SEQUENCE LISTING (5 sequences)

SEQ ID NO: 1 - DNA, 19 bp, Artificial Sequence
  Synthetic Sequence
ttactaagcacaacactct

SEQ ID NO: 2 - DNA, 19 bp, Artificial Sequence
  Synthetic sequence
tacacattactttgaatcc

SEQ ID NO: 3 - DNA, 19 bp, Artificial Sequence
  Synthetic sequence
aactgagaaactaatgcct

SEQ ID NO: 4 - DNA, 19 bp, Artificial Sequence
  Synthetic sequence
ttcacagctacatcaacct

SEQ ID NO: 5 - DNA, 19 bp, Artificial Sequence
  Synthetic Sequence
tttcttgcaaatgcattcg

(19) United States
(12) Patent Application Publication
(10) Pub. No.: US 2024/0294478 A1
(43) Pub. Date: Sep. 5, 2024

(54) ANTI-CANCER COMPOUNDS AND METHODS OF USE

(71) Applicant: YALE UNIVERSITY, New Haven, CT (US)

(72) Inventors: Seth Herzon, New Haven, CT (US); Ranjit Bindra, New Haven, CT (US); Kingson Lin, New Haven, CT (US)

(21) Appl. No.: 18/571,471

(22) PCT Filed: Jun. 17, 2022
(86) PCT No.: PCT/US2022/034036

§ 371 Date: Dec. 18, 2023

Related U.S. Application Data
(60) Provisional application No. 63/212,410, filed on Jun. 18, 2021.
(60) Provisional application No. 63/290,627, filed on Dec. 16, 2021.

(51) Int. Cl. C07D 233/90; A61K 31/4164; A61K 31/4188; A61P 35/00

(52) U.S. Cl. CPC C07D 233/90; A61K 31/4164; A61K 31/4188; A61P 35/00

(57) ABSTRACT

Disclosed are compounds having anti-cancer activity, as well as methods of using these compounds. In some instances, the compounds are useful for treating, ameliorating, and/or preventing MGMT-deficient cancers and/or MMR-deficient cancers.

### CROSS-REFERENCE TO RELATED APPLICATIONS

This patent application claims priority to U.S. Provisional Application No. 63/212,410, filed Jun. 18, 2021, and U.S. Provisional Application No. 63/290,627, filed Dec. 16, 2021, all of which are herein incorporated by reference in their entireties.

### STATEMENT REGARDING FEDERALLY SPONSORED RESEARCH OR DEVELOPMENT

This invention was made with government support under GM007205, CA215453, GM131913, and CA254158 awarded by National Institutes of Health. The government has certain rights in the invention.

### SEQUENCE LISTING

The ASCII text file named “047162-7333WO1(01712)_Seq_List_ST25” created oJun. 17, 2022, comprising 1.20 Kbytes, is hereby incorporated by reference in its entirety.

### BACKGROUND

Glioblastoma multiforme (GBM) is a malignant brain tumor with poor prognosis. It accounts for 48.3% of malignant brain and central nervous system tumors. The monofunctional alkylator temozolomide (TMZ; 4-methyl-5-oxo-2,3,4,6,8-pentazabicyclo[4.3.0]nona-2,7,9-triene-9-carboxamide) was first introduced over twenty years ago and remains the first line therapy for the treatment of patients with GBM.

However, TMZ is effective only in cells which have below normal expression of the DNA repair protein O⁶-methylguanine-DNA-methyltransferase (MGMT). These cells are termed “MGMT-deficient” or “MGMT-”. In cells which express normal levels of MGMT, termed “MGMT-proficient” or “MGMT+”, the MGNT enzyme can reverse the alkylation and restore the affected DNA to its pre-alkylation status. As approximately 50% of GB tumors are MGMT proficient, TMZ is not an effective therapy for these MGMT-proficient GBM tumors.

Furthermore, tumors also often develop mutations in mismatch repair (MMR) genes, either as a consequence of TMZ therapy or during normal tumorigenesis. These MMR deficient (MMR−) tumors are chemoresistant to TMZ therapy.

Despite over twenty years of seeking improvement on TMZ, no improved drug has emerged for the treatment of MGMT-deficient cancers. Bifunctional alkylation agents, such as lomustine (CCNU; 1-(2-chloroethyl)-3-cyclohexyl-1-nitrosourea) and mitozolomide (MTZ; 3-(2-chloroethyl)-4-oxoimidazo[5,1-d][1,2,3,5]tetrazine-8-carboxamide) have been tested with the hopes of overcoming TMZ resistance. Although CCNU and MTZ were effective in killing MGMT-deficient cancer cells, independent of MMR expression, CCNU and MTZ were found to not be suitable anti-cancer therapies owing to their high activity in, and likelihood of damaging, MGMT-proficient normal tissue cells.

### SUMMARY

In certain embodiments, the present disclosure provides compounds, compositions, and/or methods that effectively treat, ameliorate, and/or prevent tumors for which TMZ is not an effective treatment, amelioration, and/or prevention. In certain embodiments, the compounds, compositions, and/or methods of the disclosure selectively target and kill MGMT-deficient cancer cells, regardless of their level of MMR expression. In certain embodiments, the compounds, compositions, and/or methods of the disclosure avoid chemoresistance from MMR silencing. In certain embodiments, the compounds, compositions, and/or methods of the disclosure do not damage and/or kill normal tissue cells.

The present disclosure relates in one aspect to chemical compounds of formula (I)

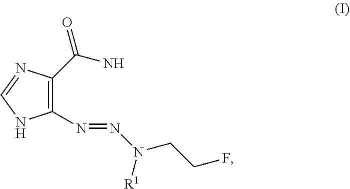

or a pharmaceutically acceptable salt thereof, wherein:

R¹ is selected from H, C₁₋₄ alkyl, hetero-substituted C₁₋₄ alkyln

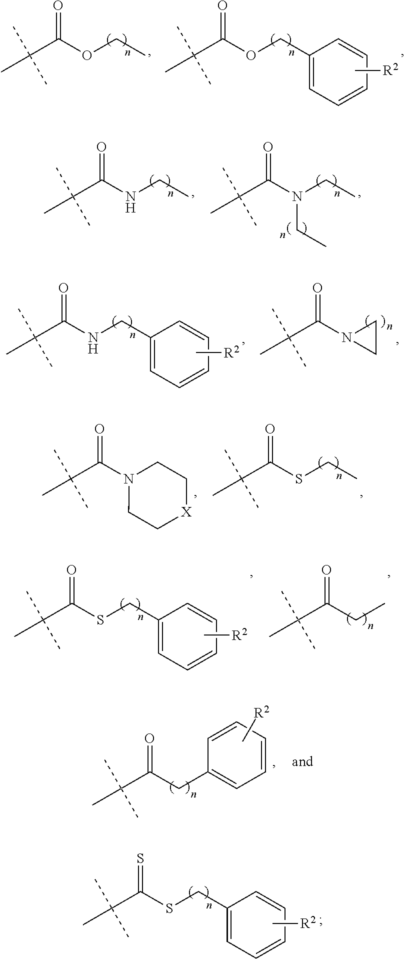

each occurrence of n is independently 0, 1, 2, 3, or 4;
X is independently selected from CH₂, NH, and 0; R² is independently selected from H, C₁₋₄ alkyl, nitro, halogen, —OC₁₋₄ alkyl, —NHC₁₋₄ alkyl, —C(O)OC₁₋₄ alkyl, and C(O)NH— C₁₋₄ alkyl; and
“hetero-substituted C₁₋₄ alkyl” is independently selected from CH₂OR³, CH(OR³)R⁴, CH₂NR³R⁴, or CH₂NC(O)R³, and
R³ and R⁴ are each independently selected from C₁₋₄ alkyl.

The present disclosure also relates in one aspect to pharmaceutical compositions comprising a compound of formula (I) or a pharmaceutically acceptable salt thereof, admixed with a pharmaceutically-acceptable carrier.

The present disclosure further relates in one aspect to method of treating, ameliorating, and/or preventing cancer in a patient in need of such treatment, amelioration, and/or prevention. In certain embodiments, the method comprises administering to the patient a therapeutically-effective amount of (1) a compound of formula (I) or a pharmaceutically acceptable salt thereof, or (2) a compound of formula (II),

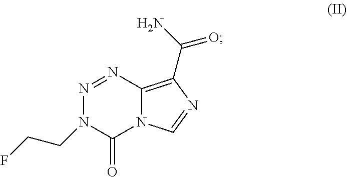 [SMILES: NC(=O)c1ncn2c(=O)n(CCF)nnc12]

or a pharmaceutically acceptable salt thereof, provided the cancer is MGMT-deficient.

### BRIEF DESCRIPTION OF THE DRAWINGS

FIGS. 1A-1G: Short-term viability assay curves for various cell lines testing the activity of KL-50 versus TMZ. FIG. 1A: Short-term viability assay curves for TMZ in DLD1 MSH6-deficient cells pre-treated with 0.01% DMSO control (CTR) or 10 μM O⁶BG (+O⁶BG) for 1 h prior to TMZ addition to deplete MGMT. FIG. 1B: Short-term viability assay curves for KL-50 in DLD1 MSH6-deficient cells pre-treated with 0.01% DMSO control (CTR) or 10 μM O⁶BG (+O⁶BG) for 1 h prior to KL-50 addition. Short-term viability assay curves for (FIG. 1C) TMZ in HCT116 MLH1−/−cells or (FIG. 1D) HCT116 cells complemented with chromosome 3 carrying wildtype MLH1 (+Chr3) pre-treated with 0.01% DMSO control or 10 μM O⁶BG (+O⁶BG) for 1 h prior to TMZ addition. Short-term viability assay curves for (FIG. 1E) KL-50 in HCT116 MLH1−/−cells or (FIG. 1F) HCT116 cells complemented with chromosome 3 carrying wildtype MLH1 (+Chr3) pre-treated with 0.01% DMSO control or 10 μM O⁶BG (+O⁶BG) for 1 h prior to KL-50 addition. FIG. 1G: Short-term cell viability curves for KL-50 and TMZ in BJ fibroblast cells (normal cells). For these figures, points, mean; error bars, SD; n>3 technical replicates.

FIGS. 2A-2C: KL-50 displays MGMT-dependent, MMR-independent cytotoxicity in clonogenic survival assays (CSAs) testing the activity of KL-50 (FIG. 2B) (wells containing 1000 plated cells treated with 30 μM KL-50) versus TMZ (FIG. 2A) (wells containing 1000 plated cells treated with 30 μM TMZ) and CCNU (FIG. 2C).

FIG. 3: Illustrative results of a clonogenic survival assay for example compound E1.

FIGS. 4A-4F: NER, BER, ROS, and altered DNA melting point do not play a major role in the mechanism of KL-50. (FIG. 4A) Short-term cell viability assays in both WT and XPA-deficient MEFs demonstrating the absence of additional sensitivity to KL-50 in NER compromised XPA deficient cells±MGMT depletion with 06BG, in contrast to cisplatin as positive control. (FIG. 4B) EndoIV depurination assay utilizing supercoiled pUC19 plasmid DNA assessing both spontaneous and enzymatically catalyzed SSB formation resulting from depurination post-treatment, demonstrating comparable levels of depurination and SSB formation by KL-50 and TMZ. (FIGS. 4C-4E) Short-term cell viability assays in LN229 MGMT+/−, MMR+/−isogenic lines pre-treated with increasing concentrations of the ROS scavenger NAC did not result in rescue of KL-50 toxicity. (FIG. 4F) Melting temperature experiments in linearized pUC19 plasmid DNA treated with 100 or 500 μM of MMS or KL-50 for 3 h resulted in comparable changes in measured DNA melting temperature. Columns, mean; error bars, SD; n=2 independent analyses. For (A) and (C), points, mean; error bars, SD; n=3 technical replicates.

FIGS. 5A-5K: KL-50 activates DNA damage response pathways and cycle arrest in MGMT-cells, independent of MMR, and induces sensitivity in cells deficient in ICL or HR repair. Phospho-SER139-H2AX (yH2AX) (FIG. 5A), 53BP1 (FIG. 5B), and phospo-SER33-RPA2 (pRPA) (FIG. 5C) foci formation quantified by % cells with 10 foci in LN229 MGMT+/−, MMR+/−cells treated with 0.1% DMSO control, 20 μM KL-50, or 20 μM TMZ for 48 h. Columns, mean; error bars, SD; n 5 technical replicates. FIGS. 5D-5K: KL-50 induces activation of the ATR-CHK1 and ATM-CHK2 signaling axes and delayed DNA repair foci formation in MGMT-deficient cells, independent of MMR status. Western blotting performed in LN229 MGMT+/−, MMR+/−cells following treatment with 20 μM KL-50 or TMZ for 24 or 48 h. Treatment with 1 μM doxorubicin for 24 h (Doxo) served as a positive control for p-CHK1 activation. Phospho-SER139-H2AX (yH2AX), 53BP1, and phospho-SER33-RPA2 (pRPA) foci levels over time following treatment with KL-50 (20 M) (FIGS. 5D-5F) or TMZ (20 μM) (FIGS. 5G-5I) for 0, 2, 8, 24, or 48 h in LN229 MGMT+/−, MMR+/−cells. Points, mean % cells with >10 foci; error bars, SD; n >5 technical replicates. (FIGS. 5J-5K) Extended time course of yH2AX foci levels following treatment with KL-50 (20 μM) or TMZ (20 μM) for 0, 48, 72, or 96 h in LN229 MGMT+/−, MMR+/−cells. Points, % cells with 10 foci, n>250 cells per condition.

FIGS. 6A-6E: Selected cell cycle analysis upon drug treatment. FIG. 6A: Percentage of cells in G1, S, and G2 cell cycle phases after treatment, as in FIGS. 5A-5C, measured using integrated nuclear (Hoechst) staining intensity. Columns, mean; error bars, SD; n=3 independent analyses. (FIGS. 6B-6E) Time course analysis of cell cycle distribution measured using integrated nuclear (Hoechst) staining intensity after treatment of LN229 MGMT+/−, MMR+/−cells with KL-50 (20 μM) or TMZ (20 μM) for 2, 8, 24, or 48 h.

DMSO (0.1%) serves as negative control and aphidicolin (10 μM) and paclitaxel (1 μM) serve as positive controls for S-phase and G2-phase arrest, respectively. Columns, mean; error bars, SD; n=3 independent analyses.

FIGS. 7A-7C: KL-50 induces DDR foci formation primarily in S and G2 cell cycle phases, and to lesser extent, in MGMT-G1 phase cells. (FIG. 7A) Phospho-SER139-H2AX (yH2AX) foci levels in LN229 MGMT+/−, MMR+/−cells in G1, S, and G2 cell cycle phases after treatment with 0.1% DMSO control, KL-50 (20 μM) or TMZ (20 μM) for 48 h. Representative foci images with nuclei labeled as G1, S, or G2 phase cells based on Hoechst staining intensity are shown on the right. (FIG. 7B) 53BP1 foci levels and representative foci images in cells treated as in (7-1). (FIG. 7C) Phospho-SER33-RPA2 (pRPA) foci levels and representative foci images in cells treated as in (7-1). For FIGS. 7A-7C, points, % cells with 10 foci; n>500 cells per condition and cell cycle phase.

FIGS. 8A-8K. (FIG. 8A) Change in percent cells with 1 micronuclei from baseline (DMSO control) after treatment as in FIGS. 5A and 5C. Columns, mean; error bars, SD; n>15 technical replicates; **** p<0.0001; ns, not significant. (FIG. 8B) Short-term viability assay curves for KL-50 in PD20 cells, deficient in FANCD2 (FANCD2−/−) or complemented with FANCD2 (+FANCD2). (FIG. 8C) Short-term viability assay curves for KL-50 in PEO4 (BRCA2+) and PEO1 (BRCA2−/−) cells pre-treated with 0.01% DMSO control or 10 μM O⁶BG (+O⁶BG) for 1 h prior to KL-50 addition. (FIG. 8D) Short-term viability assay curves for KL-50 in DLD1 BRCA2+/− and BRCA2−/−cells pre-treated with 0.01% DMSO control or 10 μM O⁶BG (+O⁶BG) for 1 h prior to KL-50 addition. For FIGS. 8B-8D, points, mean; error bars, SD; n=3 technical replicates. (FIGS. 8E-8K) Validation of micronuclei analysis, ICL sensitivity in FANCD2−/− and BRCA2−/− cell models, and demonstration of FANCD2 ubiquitination induced by KL-50. (FIG. 8E) Validation of micronuclei identification using olaparib as positive control. Change in percent cells with 1 micronuclei from baseline (DMSO control) after treatment with olaparib (10 μM) for 48 h in LN229 MGMT+/−, MMR+/−cells. Columns, mean; error bars, SD; n>15 technical replicates; **** p<0.0001. (FIGS. 8F-8G) Short-term viability assay curves for cisplatin and mitomycin (MMC) in PD20 cells, deficient in FANCD2 (FANCD2−/−) or complemented with FANCD2 (+FANCD2), demonstrating hypersensitivity to crosslinking agents in FANCD2−/−cells. (FIGS. 8H-8I) Short-term viability assay curves for cisplatin and MMC in PEO4 (BRCA2+) and PEO1 (BRCA2−/−) cells pre-treated with 0.01% DMSO control or 10 μM O⁶BG (+O⁶BG) for 1 h prior to cisplatin or MMC addition, demonstrating hypersensitivity of PEO4 BRCA2−/−cells to crosslinking agents independent of MGMT depletion. (FIGS. 8J-8K) Short-term viability assay curves for cisplatin and MMC in DLD1 BRCA2+/− and BRCA2-/− cells pre-treated with 0.01% DMSO control or 10 μM O⁶BG (+O⁶BG) for 1 h prior to cisplatin or MMC addition, demonstrating hypersensitivity of DLD1 BRCA2−/− cells to crosslinking agents independent of MGMT depletion. Western blot analysis of FANCD2 ubiquitination in LN229 MGMT+/−, MMR+/−cells and PD20 FANCD2-deficient cells, complemented with empty vector (FANCD2+EV), wildtype FANCD2 (PD20+FD2) or ubiquitination-mutant FANCD2 (PD20+KR). The % FANCD2 ubiquitination (% FANCD2 Ub) is quantified as the background-corrected integrated band intensity of the upper band divided by the sum of the background-corrected integrated band intensities of the upper and lower bands. The fold change in % FANCD2 ubiquitination is presented for each cell line relative to DMSO-treated cells. Vinculin serves as loading control. For 8J-8K, points, mean; error bars, SD; n=3 technical replicates.

FIGS. 9A-91: KL-50 is safe and efficacious on both MGMT−/MMR+ and MGMT−/MMR-flank tumors over a wide range of treatment regimens and conditions. (FIG. 9A) Xenograft LN229 MGMT−/MMR+ flank tumors treated with 3 weekly cycles of P.O. administration of 10% cyclodextrin control (n=7), TMZ (n=7, 5 mg/kg) or KL-50 (n=6, 5 mg/kg) on Monday, Wednesday, and Friday. (FIG. 9B) Xenograft LN229 MGMT−/MMR− flank tumors treated with 3 weekly cycles of P.O. administration of 10% cyclodextrin control (n=6), TMZ (n=5, 5 mg/kg) or KL-50 (n=5, 5 mg/kg) on Monday, Wednesday, and Friday. (FIG. 9C) Mean body weight of mice during LN229 flank tumor experiments. (FIG. 9D) Kaplan-Meier analysis of LN229 MGMT−/MMR− xenograft flank tumor-bearing mice to determine survival rate based on death, removal from study if mouse body weight loss exceeded 20% of initial body weight, or if tumor volume exceeded 2000 mm³. Both control and TMZ treated groups have a median OS of 10 weeks and KL-50 treated mice have median OS of greater than 15 weeks. (FIGS. 9E-9G) Xenograft LN229 MGMT−/MMR+ and LN229 MGMT−/MMR− flank tumors treated with PO administration of 10% cyclodextrin control (n=7), KL-50 (n=6, 3 cycles of 15 mg/kg on Monday, Wednesday, Friday), KL-50 (n=6, 1 cycle of 25 mg/kg Monday through Friday), or intraperitoneal (IP.) administration of KL-50 (n=7, 3 cycles of 5 mg/kg on Monday, Wednesday, Friday) revealed equal efficacy with no observable increases in toxicity as measured by mice systemic weights. (FIGS. 9H-91) Xenograft LN229 MGMT−/MMR+ and LN229 MGMT−/MSH6—flank tumors with a larger average starting tumor size of −400 mm³ and −350 mm³ respectively, treated with 3 weekly cycles of P.O administration of 10% cyclodextrin (n=4) or KL-50 (n=4, 3 cycles of 25 mg/kg on Monday, Wednesday, and Friday). The study period was limited by control groups which had to be euthanized for exceeding the ethical maximum allowed tumor size, thus ending the study. In all panels, points, mean; error bars, SEM; *, P<0.05; **, P<0.01; ***, P<0.001; ****, P<0.0001; ns, not significant.

FIGS. 10A-10G. KL-50 is efficacious in an LN229 MGMT−/MMR− intracranial model and is well tolerated with limited myelosuppression at supratherapeutic doses. (FIG. 10A) Mean tumor size as measured by bioluminescent imaging (BLI) as relative light units (RLU; photons/see) with SEM of xenograft LN229 MGMT−/MMR− intracranial tumors treated with 3 weekly cycles of P.O administration with 10% cyclodextrin control (n=10), TMZ (n=11, 25 mg/kg) or KL-50 (n=11, 25 mg/kg) on Monday, Wednesday, and Friday. (FIG. 10B) Mean body change with SEM of mice during maximum tolerated dose experiment in non-tumor bearing mice. (FIGS. 10C-10G) Complete blood counts for mice pre-treatment and 7 days post-treatment with escalations of single dose KL-50 delivered PO. WBC lower limit of normal (LLN): 2.2 K/L; Neutrophils LLN: 0.42 K/L; Lymphocyte LLN: 1.7 K/L; RBC LLN: 3.47 M/L; Platelet LLN: 155 K/L. *, P<0.05; ****, P<0.0001.

### DETAILED DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

In addition, it is to be understood that the phraseology or terminology employed herein, and not otherwise defined, is for the purpose of description only and not of limitation.

Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

The term “about” as used herein can allow for a degree of variability in a value or range, for example, within 10%, within 5%, or within 1% of a stated value or of a stated limit of a range, and includes the exact stated value or range.

The term “C₁₋₄ alkyl”, as used herein, means a linear or branched saturated hydrocarbon of 1 to 4 carbon atoms, including methyl, ethyl, propyl, isopropyl, butyl, 2-methylpropyl, and tert-butyl.

The term “halo” or “halogen”, as used herein, means a fluoro, chloro, bromo, or iodo radical.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, O-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

As used herein, the language “pharmaceutically effective amount,” “therapeutically effective amount,” or “effective amount” refers to a non-toxic but sufficient amount of the composition used in the practice of the disclosure that is effective to provide the desired biological result. That result may be reduction and/or alleviation of the signs, symptoms, or causes of a disease, or any other desired alteration of a biological system wherein a “disease” is a state of health of a patient wherein the patient cannot maintain homeostasis, and wherein if the disease is not ameliorated then the patient's health continues to deteriorate.

As used herein, the terms “patient”, “subject” and “individual” can be used interchangeably and may refer to a human or non-human mammal or a bird. Non-human mammals include, for example, livestock and pets, such as ovine, bovine, porcine, canine, feline and murine mammals. In certain embodiments, the subject is human.

As used herein, the term “treatment” or “treating” is defined as the application or administration of a therapeutic agent, i.e., a compound or compounds as described herein (alone or in combination with another pharmaceutical agent), to a patient, or application or administration of a therapeutic agent to an isolated tissue or cell line from a patient (e.g., for diagnosis or ex vivo applications), who has a condition contemplated herein or a symptom of a condition contemplated herein, with the purpose to cure, heal, alleviate, relieve, alter, remedy, ameliorate, improve or affect a condition contemplated herein, or the symptoms of a condition contemplated herein. Such treatments may be specifically tailored or modified, based on knowledge obtained from the field of pharmacogenomics.

The term “glioma”, as used herein, refers to a common type of tumor originating in the brain which originate in the glial cells that surround and support neurons in the brain, including astrocytes, oligodendrocytes and ependymal cells. A gliorna is one of the most common categories of primary brain tumor. Glioblastorna is a type of glioma.

The term “room temperature” as used herein refers to a temperature of about 15° C. to about 28° C.

### COMPOUNDS AND COMPOSITIONS

In accordance with the present disclosure are provided chemical compounds, pharmaceutical compositions comprising the compounds, and methods of treatment, amelioration, and/or prevention of cancer using the compounds and/or pharmaceutical compositions.

In various embodiments, provided herein is a compound, or a pharmaceutically acceptable salt thereof, of formula (I):

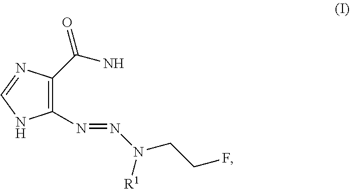

R¹ is selected from H, C₁₋₄ alkyl, hetero-substituted C₁₋₄ alkyl,

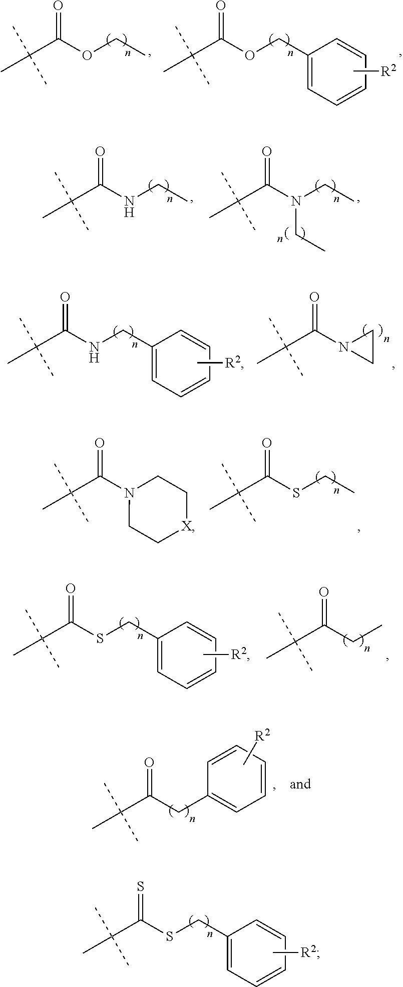

each occurrence of n is independently 0, 1, 2, 3, or 4;
X is independently selected from CH₂, NH, and 0;
R² is independently selected from H, C₁₋₄ alkyl, nitro, halogen, —OC₁₋₄ alkyl, —NHC₁₋₄ alkyl, —C(O)OC₁₋₄ alkyl, and C(O)NH— C₁₋₄ alkyl; and
“hetero-substituted C₁₋₄ alkyl” is independently selected from CH₂OR³, CH(OR³)R⁴, CH₂NR³R⁴, or CH₂NC(O)R³, and
R³ and R⁴ are each independently selected from C₁₋₄ alkyl.

In certain embodiments, R¹ is selected from hydrogen and C₁₋₄ alkyl. In certain embodiments, R¹ is H. In certain embodiments, R¹ is C₁₋₄ alkyl. In certain embodiments, R¹is methyl. In certain embodiments, R¹ is ethyl. In certain embodiments, R¹ is n-propyl. In certain embodiments, R¹ is isopropyl. In certain embodiments, R¹ is n-butyl. In certain embodiments, R¹ is sec-butyl. In certain embodiments, R¹ is iso-butyl. In certain embodiments, R¹ is tert-butyl. In certain embodiments, R¹ is hetero-substituted C₁₋₄ alkyl. In certain embodiments, R¹ is

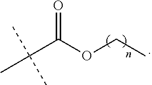

In certain embodiments, R¹ is

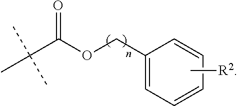

In certain embodiments, R¹ is

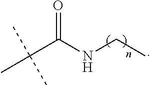

In certain embodiments, R¹ is

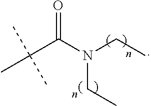

In certain embodiments, R¹ is

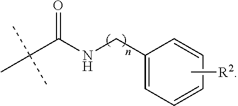

In certain embodiments, R¹ is

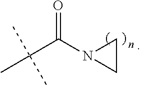

In certain embodiments, R¹ is

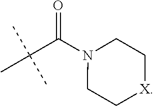

In certain embodiments, R¹ is

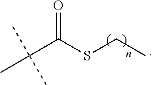

In certain embodiments, R¹ is

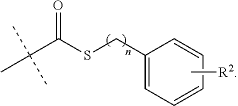

In certain embodiments, R¹ is

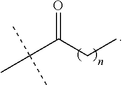

In certain embodiments, R¹ is

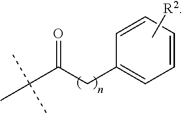

In certain embodiments, R¹ is

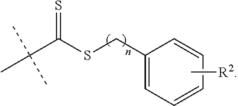

In certain embodiments, n is 0. In certain embodiments, n is 1. In certain embodiments, n is 2. In certain embodiments, n is 3. In certain embodiments, n is 4.

In certain embodiments, X is CH₂. In certain embodiments, X is NH. In certain embodiments, X is O.

In certain embodiments, R² is H. In certain embodiments, R² is C₁₋₄ alkyl. In certain embodiments, R² is nitro. In certain embodiments, R² is halogen. In certain embodiments, R² is —OC₁₋₄ alkyl. In certain embodiments, R² is —NHC₁₋₄ alkyl. In certain embodiments, R² is —C(O)OC₁₋₄ alkyl. In certain embodiments, R² is C(O)NH— C₁₋₄ alkyl.

In certain embodiments, the hetero-substituted C₁₋₄ alkyl is CH₂OR³. In certain embodiments, the hetero-substituted C₁₋₄ alkyl is CH(OR³)R⁴. In certain embodiments, the hetero-substituted C₁₋₄ alkyl is CH₂NR³R⁴. In certain embodiments, the hetero-substituted C₁₋₄ alkyl is CH₂NC(O)R³.

In certain embodiments, R³ is methyl. In certain embodiments, R³ is ethyl. In certain embodiments, R³ is n-propyl. In certain embodiments, R³ is isopropyl. In certain embodiments, R³ is n-butyl. In certain embodiments, R³ is sec-butyl. In certain embodiments, R³ is iso-butyl. In certain embodiments, R³ is tert-butyl.

In certain embodiments, R⁴ is methyl. In certain embodiments, R⁴ is ethyl. In certain embodiments, R⁴ is n-propyl. In certain embodiments, R⁴ is isopropyl. In certain embodiments, R⁴ is n-butyl. In certain embodiments, R⁴ is sec-butyl. In certain embodiments, R⁴ is iso-butyl. In certain embodiments, R⁴ is tert-butyl.

In various embodiments, provided herein is a pharmaceutical composition that includes at least one compound of formula (I) and at least one pharmaceutically acceptable carrier. In various embodiments, provided herein is a pharmaceutical composition that includes at least one compound of formula (II) and at least one pharmaceutically acceptable carrier.

The compounds of the present disclosure, and their pharmaceutically acceptable salts, shall include all their forms including hydrates, solvates, clathrates and other complexes, isomers, crystalline and non-crystalline forms, isomorphs, polymorphs, tautomers and metabolites thereof.

The compounds described herein may form salts with acids or bases, and such salts are included in the present disclosure. In certain embodiments, the salts are pharmaceutically acceptable salts. The term “salts” embraces addition salts of free acids or free bases that are compositions of the disclosure. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present disclosure, such as for example utility in process of synthesis, purification or formulation of compositions of the disclosure.

Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

In certain embodiments, compounds of the present disclosure, and salts thereof, can exist as tautomers. All tautomers are included within the scope of the present disclosure.

These compounds, and salts thereof, may exist as clathrates or other complexes. Included within the scope of the disclosure are complexes such as clathrates, drug-host inclusion complexes wherein the drug and host are present in stoichiometric or non-stoichiometric amounts. Also included are complexes containing two or more organic and/or inorganic components which may be in stoichiometric or non-stoichiometric amounts. The resulting complexes may be ionized, partially ionized, or non-ionized. For a review of such complexes, see J. Pharm. Sci., 64 (8), 1269-1288 by Haleblian (August 1975).

In certain embodiments, the compounds, and salts thereof, are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form.

The compounds, and salts thereof, also include isotopic labels wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, “C, ¹³C, ¹⁴C, ³⁶Cl, ¹⁸F, 1231 125I ¹³N, ¹⁵N, 150, 170, 180 ³²P, and ³⁵S.

In certain embodiments, the compounds described herein are labeled by other means, including, but not limited to, the use of chromophores or fluorescent moieties, bioluminescent labels, or chemiluminescent labels.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from allyl, Bn, Cbz, Alloc, ethyl, t-butyl, TBDMS, Teoc, Boc, PMB, trityl, acetyl and FMOC. Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

The compounds of formula (I) may be synthesized according to the following scheme:

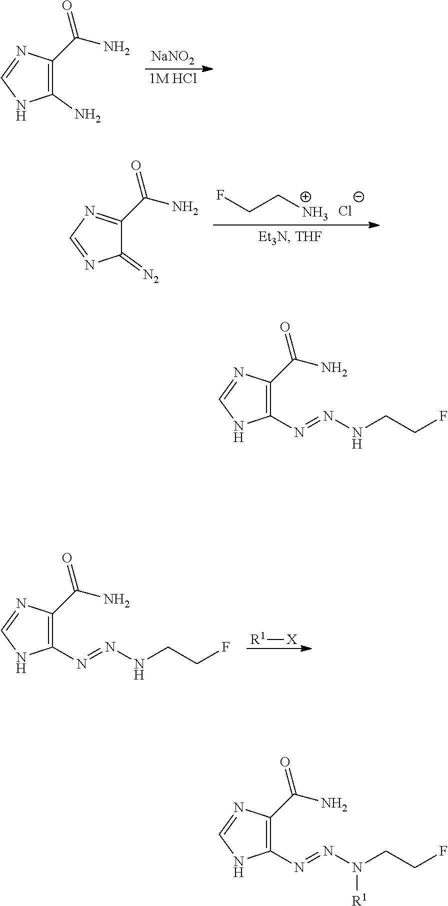

The starting materials are commercially available, as is N,N-methyl(2-fluoroethyl)amine.

The compounds of the present disclosure may be prepared according to the following synthetic conditions: To a flame dried round bottom flask is added 4-diazo-4H-imidazole-5-carboxamide (1 equiv.) in [0.15] M THF under magnetic stirring at room temperature under inert atmosphere. The reaction vessel is then charged with 1.2 equivalent of the amine (F—CH₂—CH₂—NHR₁), with the addition of 1.2 equivalents of sacrificial base if using the conjugate acid of the amine. The reaction proceeds for 3-10 hours before filtration and washing with ethyl acetate followed by diethyl ether.

### PHARMACEUTICAL COMPOSITIONS AND FORMULATIONS

Pharmaceutical compositions suitable for use in the methods described herein, and salts thereof, can include one or more of the disclosed compounds and a pharmaceutically acceptable carrier or diluent. The composition may be formulated for intravenous, subcutaneous, intraperitoneal, intramuscular, topical, oral, buckle, nasal, pulmonary or inhalation, ocular, vaginal, or rectal administration. In some embodiments, the compounds are formulated for oral administration. The pharmaceutical composition can be formulated to be an immediate-release composition, sustained-release composition, delayed-release composition, etc., using techniques known in the art.

Pharmacologically acceptable carriers for various dosage forms are known in the art. For example, excipients, lubricants, binders, and disintegrants for solid preparations are known; solvents, solubilizing agents, suspending agents, isotonicity agents, buffers, and soothing agents for liquid preparations are known. In some embodiments, the pharmaceutical compositions include one or more additional components, such as one or more preservatives, antioxidants, stabilizing agents and the like.

Additionally, the disclosed pharmaceutical compositions can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In some embodiment, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Pharmaceutical compositions of the disclosure can be administered in combination with other therapeutics that are part of the current standard of care for cancer. METHODS In the present disclosure, at least one compound or pharmaceutical compositions is administered to a patient (e.g., a human patient) suffering from cancer.

In certain embodiments, the cancer is cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, leukemia, urothelial cancer, colorectal cancer, and/or glioblastoma multiforme.

In certain embodiments, the cancer is a breast invasive carcinoma, colon adenocarcinoma, head and neck cancer, lung adenocarcinoma, rectal adenocarcinoma, acute myeloid leukemia, glioblastoma multiforme, brain lower grade glioma, colorectal cancer, or metastatic melanoma. In certain embodiments, the cancer is a melanoma.

In certain embodiments, the disorder is a cancer selected from the group consisting of ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, and leukemia.

In certain embodiments, the cancer is a solid tumor. In certain embodiments, the cancer is a sarcoma or carcinoma. In certain embodiments, the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, or leukemia.

In certain embodiments, the cancer is prostate cancer, breast cancer, lung cancer, liver cancer, bladder cancer, urinary tract cancer, or eye cancer. In certain embodiments, the cancer is prostate cancer. In certain embodiments, the cancer is breast cancer. In certain embodiments, the cancer is lung cancer. In certain embodiments, the cancer is liver cancer. In certain embodiments, the cancer is bladder cancer. In certain embodiments, the cancer is urinary tract cancer. In certain embodiments, the cancer is eye cancer.

In certain embodiments, the cancer is squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas (e.g., Burkitt's lymphoma and Non-Hodgkin's lymphoma); benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas.

In certain embodiments, the cancer is a neuroblastoma, craniopharyngioma, glioma, glioblastoma, schwannoma, astrocytoma, oligodendroglioma, medulloblastoma, pinealoma, hemangioblastoma, retinoblastoma, ependymoma, chordoma, meningioma, medullary carcinoma, small cell lung carcinoma, papillary adenocarcinoma, papillary carcinoma, mesothelioma, nasopharyngeal carcinoma, acoustic neuroma, oral cancer, esophageal cancer, head and neck cancer, stomach cancer, colon cancer, rectal cancer, skin cancer, melanoma, sweat gland carcinoma, sebaceous gland carcinoma, squamous cell carcinoma, basal cell carcinoma, bile duct cancer, gallbladder cancer, liver cancer, hepatocellular carcinoma, pancreatic cancer, bladder carcinoma, renal cell carcinoma, kidney cancer, Wilms' tumor, thyroid cancer, parathyroid tumor, synovioma, soft tissue sarcoma (e.g., rhabdomyosarcoma (RMS)), Kaposi sarcoma, synovial sarcoma, osteosarcoma, Ewing's sarcoma, malignant rhabdoid tumor, leiomyosarcoma, liposarcoma, lymphangioendothelio-sarcoma, lymphangiosarcoma, myxosarcoma, osteogenic sarcoma, fibrosarcoma, chondrosarcoma, or endotheliosarcoma.

In certain embodiments, the cancer is a lymphoma. In certain embodiments, the cancer is Burkitt's lymphoma, diffuse large B-cell lymphoma (DLBCL), follicular lymphoma, non-Hodgkin's lymphoma, lymphoid malignancies of T-cell or B-cell origin, peripheral T-cell lymphoma, adult T-cell leukemia-lymphoma, or Waldenstrom's macroglobulinemia.

In certain embodiments, the cancer is a leukemia. In certain embodiments, the cancer is acute leukemia, lymphoblastic leukemia, acute lymphoblastic leukemia, myelogenous leukemia, acute myelogenous leukemia, acute T-cell leukemia, chronic leukemia, chronic lymphocytic leukemia, chronic myelocytic leukemia, chronic myelogenous leukemia, polycythemia vera, multiple myeloma, or erythroleukemia.

In certain embodiments, the cancer is a myelodysplastic and/or myeloproliferative syndrome. In certain embodiments, the cancer is a myelodysplastic syndrome. In certain embodiments, the cancer is a myeloproliferative syndrome.

In certain embodiments, the cancer is a cancer or related myeloproliferative disorder selected from histiocytosis, essential thrombocythemia, myelofibrosis, heavy chain disease, and other malignancies and hyperproliferative disorders of the bladder, breast, colon, lung, ovaries, pancreas, prostate, skin and uterus.

In certain embodiments, the cancer is a B-cell non-Hodgkin's lymphoma, advanced solid tumor, soft tissue sarcoma, INIl-deficient cancer, BAP1-deficient cancer, follicular lymphoma, relapsed/refractory follicular lymphoma, diffuse large B-cell lymphoma, relapsed/refractory diffuse large B-cell lymphoma, non-Hodgkin's lymphoma, pediatric non-Hodgkin's lymphoma, pediatric non-Hodgkin's lymphoma with EZH2, SMARCB], or SMARCA4 mutation, histiocytic disorder, pediatric histiocytic disorder, pediatric histiocytic disorder with EZH2, SMARCB], or SMARCA4 mutation, solid tumor with EZH2, SMARCB], or SMARCA4 mutation, resistant prostate cancer, relapsed/refractory small-cell lung carcinoma, B-cell lymphoma, relapsed/refractory B-cell lymphoma, adult T-cell leukemia-lymphoma, or advanced diffuse large B-cell lymphoma.

In certain embodiments, the cancer is a malignant rhabdoid tumor, atypical teratoid rhabdoid tumor, epithelioid sarcoma, renal medullary carcinoma, pancreatic undifferentiated rhabdoid carcinoma, schwannoma, epithelioid malignant peripheral nerve sheath tumor, or diffuse intrinsic glioma.

In certain embodiments, the cancer is retinoblastoma multiforme, metastatic castration-resistant prostate cancer, prostate small cell neuroendocrine carcinoma, small-cell lung cancer, triple-negative breast cancer, hepatocellular carcinoma, bladder cancer, or urinary tract cancer.

In certain embodiments, the cancer is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, epithelial carcinoma, glioma, astrocytoma, medulloblastoma, and hemangioblastoma. In certain embodiments, the cancer is a neuroblastoma, meningioma, hemangiopericytoma, multiple brain metastase, glioblastoma multiforms, glioblastoma, brain stem glioma, poor prognosis malignant brain tumor, malignant glioma, anaplastic astrocytoma, anaplastic oligodendroglioma, neuroendocrine tumor, rectal adeno carcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma, localized melanoma, malignant mesothelioma, malignant pleural effusion mesothelioma syndrome, peritoneal carcinoma, papillary serous carcinoma, gynecologic sarcoma, soft tissue sarcoma, scelroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waidenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, or leiomyoma.

In certain embodiments, the cancer is a metastatic cancer. In certain embodiments, the cancer is a relapsed and/or refractory cancer.

In certain embodiments, the cancer is ovarian cancer, uterine cancer, gestational trophoblastic disease, endometrial cancer, cervical cancer, embryonal carcinoma, choriocarcinoma, prostate cancer (including hormone insensitive and castrate resistant prostate cancers), testicular tumors (including germ cell testicular cancer/seminoma), cystadenocarcinoma, breast cancer (including estrogen-receptor positive breast cancer), brain tumors (including neuroblastoma, craniopharyngioma, glioma, glioblastoma, schwannoma, astrocytoma, oligodendroglioma, medulloblastoma, and pinealoma), hemangioblastoma, retinoblastoma, ependymoma, chordoma, meningioma, medullary carcinoma, lung cancer (including small cell lung carcinoma, papillary adenocarcinomas, and papillary carcinoma), mesothelioma, nasopharyngeal carcinoma, acoustic neuroma, oral cancer, esophageal cancer, head and neck cancer, stomach cancer, colon cancer, rectal cancer, skin cancer, melanoma, sweat gland carcinoma, sebaceous gland carcinoma, squamous cell carcinoma, basal cell carcinoma, bile duct cancer, gallbladder cancer, liver cancer, hepatocellular carcinoma, pancreatic cancer, bladder carcinoma, renal cell carcinoma, kidney cancer, Wilms' tumor, thyroid cancer, parathyroid tumor, synovioma, soft tissue sarcoma (e.g., rhabdomyosarcoma (RMS)), Kaposi sarcoma, synovial sarcoma, osteosarcoma, Ewing's sarcoma, malignant rhabdoid tumor, leiomyosarcoma, liposarcoma, lymphangioendothelio-sarcoma, lymphangiosarcoma, myxosarcoma, osteogenic sarcoma, fibrosarcoma, chondrosarcoma, or endotheliosarcoma.

In certain embodiments, the cancer is glioblastoma multiforme, brain lower grade glioma, bladder urothelial carcinoma, breast invasive carcinoma, colon adenocarcinoma, lung adenocarcinoma, lung squamous cell carcinoma, rectum adenocarcinoma, head tumor, neck tumor, gastric cancer, pancreatic cancer, or acute myeloid leukemia.

In various embodiments, a method of treating, ameliorating, and/or preventing cancer in a patient in need thereof includes administering to the patient a therapeutically-effective dose of a compound, or a pharmaceutically acceptable salt thereof, of formula (I), and/or a compound, or a pharmaceutically acceptable salt thereof, of formula (II):

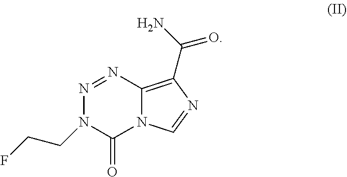 [SMILES: NC(=O)c1ncn2c(=O)n(CCF)nnc12]

In certain embodiments, the cancer is a glioma.

In certain embodiments, the cancer is MGMT-deficient (MGMT−). In certain embodiments, the cancer is both MGMT-deficient and MMR-deficient (MGMT−/MMR−).

In yet other embodiments, the cancer is resistant to temozolomide.

In certain embodiments, the method of treating, ameliorating, and/or preventing cancer with KL-50, or salts thereof, does not kill MGMT-proficient cells, such as but not limited to normal tissue cells.

The methods described herein include administering to the subject a therapeutically effective amount of KL-50, or a salt thereof, which is optionally formulated in a pharmaceutical composition. The methods described herein also include administering to the subject a therapeutically effective amount of at least, greater than, or equal to about 95, 96, 97, 98, 99, 99.9, or 99.99% pure Polymorph I of KL-50, or a salt thereof, which is optionally formulated in a pharmaceutical composition. The methods described herein also include administering to the subject a therapeutically effective amount of at least, greater than, or equal to about 95, 96, 97, 98, 99, 99.9, or 99.99% pure Polymorph II of KL-50, or a salt thereof, which is optionally formulated in a pharmaceutical composition. In various embodiments, the therapeutically effective amount of KL-50, or salts thereof, present in a pharmaceutical composition is the only therapeutically active compound in a pharmaceutical composition. In certain embodiments, the method further comprises administering to the subject an additional therapeutic agent that treats, ameliorates, and/or prevents, or aids in treating, ameliorating, and/or preventing, cancer.

In certain embodiments, the method of the present disclosure further comprises determining whether the cancer is MGMT-deficient using any detection method known in the art such as methylation-specific PCR.

In some embodiments, the therapeutically effective amount of the compound is administered together with a pharmaceutically acceptable carrier. Suitable pharmaceutically acceptable carriers are well-known in the art, as discussed elsewhere herein. A typical route of administration is oral, but other routes of administration are possible, as is well understood by those skilled in the medical arts. Administration may be by single or multiple doses. The amount of compound administered and the frequency of dosing may be optimized by the physician for the particular patient. In various embodiments, any of the dosage forms described herein can be in a unit dose form. Unit form doses as used herein refers to physically discrete units suitable as unitary dosages for the patients to be treated; each unit containing a predetermined quantity of therapeutic compound calculated to produce the desired therapeutic effect in association with the required pharmaceutical vehicle or carrier.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY—P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Suitable dispersing agents include, but are not limited to, potato starch, sodium starch glycollate, poloxamer 407, or poloxamer 188. One or more dispersing agents can each be individually present in the composition in an amount of about 0.01% w/w to about 90% w/w relative to weight of the dosage form. One or more dispersing agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Surface-active agents (surfactants) include cationic, anionic, or non-ionic surfactants, or combinations thereof Suitable surfactants include, but are not limited to, behentrimonium chloride, benzalkonium chloride, benzethonium chloride, benzododecinium bromide, carbethopendecinium bromide, cetalkonium chloride, cetrimonium bromide, cetrimonium chloride, cetylpyridine chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, tetramethylammonium hydroxide, thonzonium bromide, stearalkonium chloride, octenidine dihydrochloride, olaflur, N-oleyl-1,3-propanediamine, 2-acrylamido-2-methylpropane sulfonic acid, alkylbenzene sulfonates, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium laurate, sodium laureth sulfate, sodium lauroyl sarcosinate, sodium myreth sulfate, sodium nonanoyloxybenzenesulfonate, sodium pareth sulfate, sodium stearate, sodium sulfosuccinate esters, cetomacrogol 1000, cetostearyl alcohol, cetyl alcohol, cocamide diethanolamine, cocamide monoethanolamine, decyl glucoside, decyl polyglucose, glycerol monostearate, octylphenoxypolyethoxyethanol CA-630, isoceteth-20, lauryl glucoside, octylphenoxypolyethoxyethanol P-40, Nonoxynol-9, Nonoxynols, nonyl phenoxypolyethoxylethanol (NP-40), octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80. One or more surfactants can each be individually present in the composition in an amount of about 0.01% w/w to about 90% w/w relative to weight of the dosage form. One or more surfactants can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable diluents include, but are not limited to, calcium carbonate, magnesium carbonate, magnesium oxide, sodium carbonate, lactose, microcrystalline cellulose, calcium phosphate, calcium hydrogen phosphate, and sodium phosphate, Cellactose® 80 (75% a-lactose monohydrate and 25% cellulose powder), mannitol, pre-gelatinized starch, starch, sucrose, sodium chloride, talc, anhydrous lactose, and granulated lactose. One or more diluents can each be individually present in the composition in an amount of about 0.01% w/w to about 90% w/w relative to weight of the dosage form. One or more diluents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable granulating and disintegrating agents include, but are not limited to, sucrose, copovidone, corn starch, microcrystalline cellulose, methyl cellulose, sodium starch glycollate, pregelatinized starch, povidone, sodium carboxy methyl cellulose, sodium alginate, citric acid, croscarmellose sodium, cellulose, carboxymethylcellulose calcium, colloidal silicone dioxide, crosspovidone and alginic acid. One or more granulating or disintegrating agents can each be individually present in the composition in an amount of about 0.01% w/w to about 90% w/w relative to weight of the dosage form. One or more granulating or disintegrating agents can each be individually present in the composition in an amount of at least, greater than, or less than about 0.01%, 0.05%, 0.1%, 0.5%, 1%, 2%, 3%, 4%, 5%, 10, 1%, 20%, 25%, 30%, 35%,0 40%, 45% 50%, 55%, 60%, 65%, 70%, 75%, 80%, 85%, or 90% w/w relative to weight of the dosage form.

Suitable binding agents include, but are not limited to, gelatin, acacia, pre-gelatinized maize starch, polyvinylpyrrolidone, anhydrous lactose, lactose monohydrate, hydroxypropyl methylcellulose, methylcellulose, povidone, polyacrylamides, sucrose, dextrose, maltose, gelatin, polyethylene glycol. One or more binding agents can each be individually present in the composition in an amount of about 0.01% w/w to about 90% w/w relative to weight of the dosage form. One or more binding agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable lubricating agents include, but are not limited to, magnesium stearate, calcium stearate, hydrogenated castor oil, glyceryl monostearate, glyceryl behenate, mineral oil, polyethylene glycol, poloxamer 407, poloxamer 188, sodium laureth sulfate, sodium benzoate, stearic acid, sodium stearyl fumarate, silica, and talc. One or more lubricating agents can each be individually present in the composition in an amount of about 0.01% w/w to about 90% w/w relative to weight of the dosage form. One or more lubricating agents can each be individually present in the composition in an amount of at least, greater than, or less than about 0.01%, 0.05^{%}, 0.1%, 0.5%, ₁%, 2%, 3%, 4%, 5%, 10, 1%, 20%, 25%, 30%, 35%, 40%, 45%, 50%, 55%, 60%, 65%, 70%, 75%, 80%, 85%, or 90% w/w relative to weight of the dosage form.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing a compound as described herein. The coating can contain, for example, EUDRAGIT® L, S, FS, and/or E polymers with acidic or alkaline groups to allow release of a compound as described herein in a particular location, including in any desired section(s) of the intestine. The coating can also contain, for example, EUDRAGIT® RL and/or RS polymers with cationic or neutral groups to allow for time controlled release of a compound as described herein by pH-independent swelling.

Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as such as lauryl, stearyl, or oleyl alcohols, or similar alcohol.

Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### CONTROLLED RELEASE FORMULATIONS AND DRUG DELIVERY SYSTEMS

In certain embodiments, the compositions and formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In certain embodiments, the compound(s) described herein are administered to a patient, alone or in combination with another pharmaceutical agent, using a sustained release formulation. In certain embodiments, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Therapeutically Effective Doses and Dosing Regimens

In some embodiments, the therapeutically effective dose of the compound may be administered every day, for 21 days followed by a 7 day rest, every 7 days with a 7 day rest in between each dosage period, or for 5 continuous days followed by a 21 day rest, in each instance referring to a 28 day dosage cycle.

The therapeutically effective dose of compound administered to the patient (whether administered in a single does or multiple doses) should be sufficient to treat, ameliorate, and/or prevent the cancer. Such therapeutically effective amount may be determined by evaluating the symptomatic changes in the patient.

Exemplary doses can vary according to the size and health of the individual being treated, the condition being treated, and the dosage regimen adopted. In some embodiments, the effective amount of a disclosed compound per 28 day dosage cycle is about 1.5 g/m²; however, in some situations the dose may be higher or lower—for example 2.0 g/m² or 1.0 g/m². The daily dose may vary depending on (inter alia) the dosage regimen adopted. For example, if the regimen is dosing for five days followed by a 21 day rest and the total dosage per 28 day cycle is 1.0 g/m², then the daily dose would be 200 mg/m². Alternatively, if the regimen is dosing for 21 days followed by a 5 day rest and the total dosage per 28 day cycle is 1.6 g/m², then the daily dose would be 75 mg/m². Similar results would obtain for other dosage regimens and total 28 day doses.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

The disclosed methods of treatment may also be combined with other known methods of treatment as the situation may require.

Therapeutically effective doses and dosing regimens of the foregoing methods may vary, as would be readily understood by those of skill in the art. Dosage regimens may be adjusted to provide the optimum desired response. For example, in some embodiments, a single bolus dose of the compound may be administered, while in some embodiments, several divided doses may be administered over time, or the dose may be proportionally reduced or increased in subsequent dosing as indicated by the situation.

### Examples

Various embodiments of the present application can be better understood by reference to the following Examples which are offered by way of illustration. The scope of the present application is not limited to the Examples given herein.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

The disclosure is now described with reference to the following Examples. These Examples are provided for the purpose of illustration only, and the disclosure is not limited to these Examples, but rather encompasses all variations that are evident as a result of the teachings provided herein.

Although the compounds, compositions and methods of the disclosure have been described in the present disclosure by way of illustrative examples, it is to be understood that the disclosure is not limited thereto and that variations can be made as known by those skilled in the art without departing from the teachings of the disclosure defined by the appended claims.

Compounds:

Compounds of the present disclosure were synthesized as follows.

Preparation of Chemical Intermediate 4-diazo-4H-imidazole-5-carboxamide Commercially available 5-aminoimidazole-4-carboxamide HCl (0.78 g, 4.81 mmol) (Sigma-Aldrich) was dissolved in 8 mL aqueous 1 M HCl. This solution was then added dropwise to a stirring solution of NaNO₂ (0.37 g, 5.32 mmol) in water (8 mL) at 0° C. over the course of 1 minute. The reaction was allowed to stir for 10 minutes, after which a precipitate formed. This precipitate was filtered and washed with water followed by lyophilization overnight to yield the dry 4-diazo-4H-imidazole-5-carboxamide (0.4 g, 60% yield).

### Example 1: Synthesis of Compound E1

### 5-[(1E)-3-(2-fluoroethyl)triaz-1-en-1-yl]-1H-imidazole-4-carboxamide

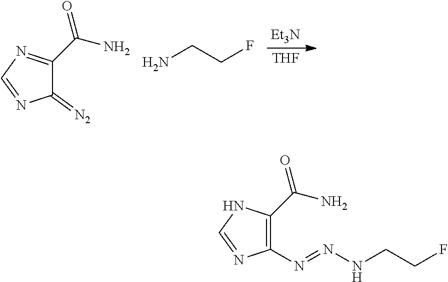

To a flame dried 50 mL round bottom flask under nitrogen was added 4-diazo-4H-imidazole-5-carboxamide (250 mg, 1.82 mmol) and charged with 15 mL dry tetrahydrofuran (THF). The reaction vessel was then charged with 2-fluoroethylamine hydrochloride (200 mg, 2 mmol) and triethylamine (280 μL, 2 mmol). The reaction was allowed to run at room temperature for 6 hours. The precipitate was then filtered and washed sequentially with ethyl acetate and diethyl ether to yield 5-[(1E)-3-(2-fluoroethyl)triaz-1-en-1-yl]-1H-imidazole-4-carboxamide (226 mg, 62% yield).

¹H NMR (500 MHz, DMSO-d₆) δ 12.62 (s, 1H), 10.88 (s, 1H), 7.51 (s, 1H), 7.41 (s, 1H), 7.16 (s, 1H), 4.64 (d, J=47.0 Hz, 2H), 3.77 (d, J=29.0 Hz, 2H).
¹⁹F NMR (376 MHz, DMSO-d₆) 6-219.91 (tt, J=48.6, 26.9 Hz).
¹³C NMR (151 MHz, DMSO-d₆) δ 161.71, 155.83, 150.17, 53.93, 18.49, 17.17.
HRMS ESI MS m/z calcd. For C6H9FN6O ([M+H]+) 200.0822; found 201.0892.

### Example 2: Synthesis of Compound E2

### (E)-5-(3-(2-fluoroethyl)-3-(2-phenylacetyl) triaz-1-en-1-yl)-1H-imidazole-4-carboxamide

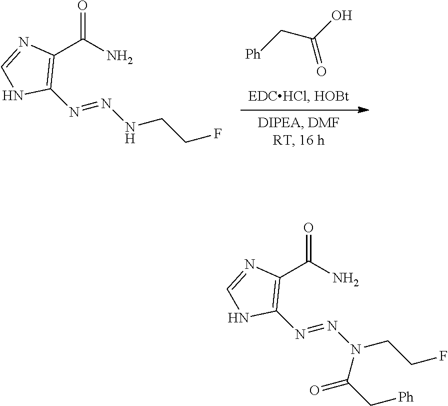 [SMILES: NC(=O)c1nc[nH]c1/N=N/NCCF ; O=C(O)Cc1ccccc1 ; NC(=O)c1nc[nH]c1/N=N/N(CCF)C(=O)Cc1ccccc1]

To a stirred solution of compound E1 (1.0 g, 4.99 mmol, 1.0 eq) in dimethylformamide (DMF) (10 mL) were added Phenylacetic acid (679 mg, 4.99 mmol, 1 eq), EDC. HCl (1.14 g, 5.98 mmol, 1.2 eq), DIPEA (1.76 mL, 9.98 mmol, 2.0 eq) and HOBt (674 mg, 4.99 mmol, 1.0 eq) at room temperature and stirred for 16 h. Progress of the reaction was monitored by TLC. After completion of starting material, the reaction mixture was diluted with water (60 mL) and extracted ethyl acetate (3×40 mL). The combined organic extracts were washed with water (25 mL), brine (25 mL) and dried over anhydrous Na2SO4, filtered, and concentrated under vacuum to obtained crude compound which was purified by flash chromatography by eluting with 25% MeOH in dichloromethane (DCM) to afford 250 mg of compound which was triturated with dichloromethane (2×10 mL) and ethyl acetate (10 mL) and dried under vacuum to afford the titled compound as pale brown solid (52 mg, 3.2%).

¹H NMR (400 MHz, DMSO-d₆) δ 13.22 (s, 1H) 7.76-7.88 (m, 2H) 7.39 (br d, J=6.85 Hz, 2H) 7.20-7.34 (m, 3H) 7.05 (s, 1H) 4.63 (br t, J=4.65 Hz, 1H) 4.51 (br d, J=4.89 Hz, 1H) 4.48 (br d, J=4.89 Hz, 1H) 4.41 (br d, J=4.40 Hz, 1H) 4.24 (s, 2H); LCMS: 96.25%; ESI MS m/z calcd. For C14H15FN602 ([M+H]+) 318.2; found 319.2.

### Example 3: Synthesis of Compound E3

### (E)-5-(3-acetyl-3-(2-fluoroethyl)triaz-1-en-1-yl)-1H-imidazole-4-carboxamide

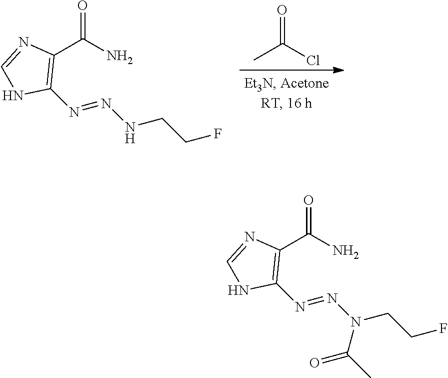

To a stirred solution of compound E1 (1.0 g, 5 mmol) in acetone (100 mL) were added triethylamine (Et3N) (1.39 mL, 10 mmol, 2 eq) and acetyl chloride (431 mg, 5.5 mmol, 1.1 eq) at room temperature and stirred for 16 h. Progress of the reaction was monitored by TLC. After completion of starting material, solvents were evaporated under vacuum to obtain crude compound which was purified by flash chromatography by eluting with 5% MeOH in ethyl acetate. The combined eluents were concentrated and triturated with ethyl acetate and the solid was washed with heptane (20 mL) and dried under vacuum to afford the titled compound as pale yellow solid (77 mg, 6.3%).

¹H NMR (400 MHz, DMSO-d₆) δ 13.17 (br s, 1H) 7.76 (s, 2H) 7.04 (br s, 1H) 4.64 (br t, J=4.65 Hz, 1H) 4.51 (br d, J=4.89 Hz, 1H) 4.45 (br s, 1H) 4.39 (br s, 1H) 2.47 (s, 3H); LCMS: 99.64%; ESI MS m/z caled. For CSHiiFN6O2 [M+H)±242.2; found 243.2.

### Example 4: Synthesis of Compound E4

### (E)-5-(3-acetyl-3-(2-fluoroethyl) triaz-1-en-1-yl)-1H-imidazole-4-carboxamide

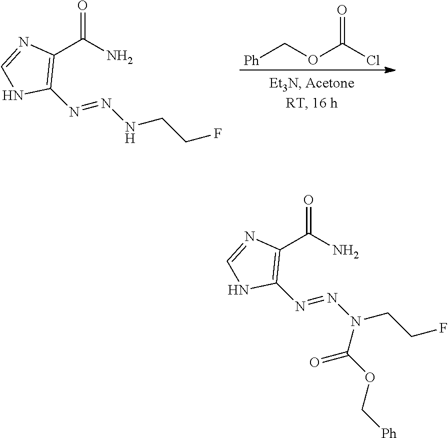 [SMILES: NC(=O)c1nc[nH]c1/N=N/NCCF ; O=C(Cl)OCc1ccccc1 ; NC(=O)c1nc[nH]c1/N=N/N(CCF)C(=O)OCc1ccccc1]

To a stirred solution of compound E1 (1.0 g, 5 mmol, 1.0 eq) in acetone (200 mL) were added Et₃N (1.38 mL, 10 mmol, 2 eq) and benzyl chloroformate (1.70 mL, 5 mmol, 50% in toluene) at room temperature and stirred for 16 h. Progress of the reaction was monitored by TLC. After completion of starting material, solvents were evaporated under vacuum to obtain crude compound which was purified by flash chromatography by eluting with 2% methanol in ethyl acetate, this purification was repeated thrice in the same method to afford the titled compound as pale-yellow solid (42 mg, 2.5%).

¹H NMR (400 MHz, DMSO-d₆) δ 13.13 (br s, 1H) 7.74 (s, 2H) 7.44-7.50 (m, 2H) 7.33-7.45 (m, 3H) 7.25 (br s, 1H) 5.39 (s, 2H) 4.68 (br t, J=4.65 Hz, 1H) 4.52-4.60 (m, 1H) 4.47 (br s, 1H) 4.41 (br d, J=3.91 Hz, 1H); LCMS: 95.77%; ESI MS m/z calcd. For C14H15FN603 ([M+H]+) 334.2; found 335.2.

### Example 5: Synthesis of Compound E5

### 4-[(1E)-3-(2-fluoroethyl)-3-methyltriaz-1-en-1-yl]-1H-imidazole-5-carboxamide

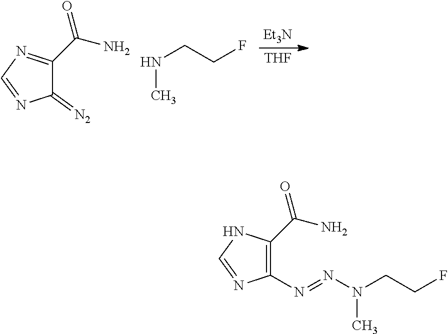

To a flame dried 50 mL round bottom flask under nitrogen was added 4-diazo-4H-imidazole-5-carboxamide (250 mg, 1.82 mmol) and charged with 15 mL dry THF. The reaction vessel was then charged with (2-fluoroethyl)-methyl-amine trifluoroacetic acid (382 mg, 2 mmol) and triethylamine (280 μL, 2 mmol). The reaction was allowed to run at room temperature for 6 hours. The precipitate was then filtered and washed sequentially with ethyl acetate and diethyl ether to yield 4-[(1E)-3-(2-fluoroethyl)-3-methyltriaz-1-en-1-yl]-1H-imidazole-5-carboxamide as the trifluoroacetate salt (324.5 mg, 55% yield). 1H NMR (500 MHz, DMSO-d₆) δ 12.64 (s, 1H), 8.47 (s, 1H), 7.51 (s, 1H), 7.44 (s, 1H), 7.25 (s, 1H), 5.53-3.80 (m, 4H), 3.24 (d, J=62.2 Hz, 3H). ¹⁹F NMR (471 MHz, DMSO-d₆) 6-222.01-1-222.78 (m).

Biological Activity: The compounds E1-E5 and KL-50 were evaluated for their biological activity against various cancer cells, with comparison to one or more known anti-cancer agents, as follows.

### Example 6: Short-term Cell Viability Assays

The cytotoxicity of the Compounds E1-E5, and of TMZ, were evaluated in short-term cell viability assays, in vitro, against four isogenic LN229 glioblastoma cell lines engineered to be MGMT-proficient (“MGMT+”) or MGMT-deficient (MGMT−) and proficient or deficient in MMR activity (MMR+ or MMR−), using short hairpin RNAs (shRNAs) targeting MSH2 (referred to as MGMT+/−, MMR+/−cells). LN229 MGMT- and MGMT+ cell lines were a gift from B. Kaina (Johannes Gutenberg University Mainz, Mainz, Germany) and grown in Gibco® Duilbecco's Modified Eagle Medium with 10% fetal bovine serum (DMEM). For each compound tested, the relationship between MGMT status and MMR status and compound activity, in terms of IC₅₀ values (in M), was determined by the following method. On day 1, LN229 isogenic cells of varying MGMT and MMR status were seeded in 96 well format at a density of 2000 cells/well in 100 μL of DMEM media and allowed to adhere overnight. On day 2, a drug master plate was made with 100×the desired maximal concentration of test compound and serially diluted by 2 until 100×the minimal desired concentration, with one DMSO control. Then daughter plates were created with varied concentrations from 3-times the minimal concentration to 3-times the maximal desired concentration. Afterwards, 50 μL of daughter drug plate was added to 100 μL of the seeded cells for a final concentration of 1×in triplicate. Cells were allowed to grow for 6 days. On day 7, the cells were fixed, and stained with Hoechst nuclear dye and imaged to determine growth inhibition.

The IC₅₀ values of compounds E1-E5, and of the comparator alkylating agent temozolomide (TMZ) purchased from Selleck Chemicals, are shown in Table 1, below.

TABLE 1
IC₅₀ (μM) against LN229 cell lines

| | MGMT+ | MGMT+ | MGMT− | MGMT− |
|---|---|---|---|---|
| Compound | MMR+ | MMR− | MMR+ | MMR− |
| TMZ | >100 | >100 | 8.3 ± 3.5 | 838 ± 100 |
| E1 | >100 | >100 | 24 ± 2 | 22 ± 1 |
| E2 | >100 | 72 ± 30 | 28 ± 1 | 21 ± 1 |
| E3 | >100 | >100 | 40 ± 2 | 46.0 ± 4 |
| E4 | 14 ± 1 | 11 ± 1 | 8 ± 1 | 17 ± 1 |
| E5 | >199 | >100 | 14.2 ± 1 | >100 |

TMZ, which is of known efficacious use for the treatment of cancers deficient in MGMT, was found to be essentially inactive in MGMT+cells, regardless of MMR status, and also in MGMT−/MMR− cells. All of the compounds of the present disclosure were found to be active against one or more LN229 glioblastoma cell lines. In particular, Compounds E1 and E3 each demonstrated exquisite sensitivity in MGMT−/MMR− cells, with limited activity in MGMT+cells.

In addition, the cytotoxicity of the compound KL-50, as compared to known the anti-cancer agents TMZ, mitozolomide (MTZ) and lomustine (CCNU), was evaluated in short-term cell viability assays were evaluated, in vitro, against four isogenic LN229 glioblastoma cell lines engineered to be MGMT-proficient (“MGMT+”) or MGMT-deficient (MGMT−) and proficient or deficient in MMR activity (MMR+ or MMR−), using short hairpin RNAs (shRNAs) targeting MSH2 (referred to as MGMT+/−, MMR+/−cells). For each compound tested, the relationship between MGMT status and MMR status and compound activity, in terms of IC₅₀ values (in μM), was determined. In addition, for each compound, the therapeutic index (TI) was calculated, according to the equation MGMT TI=IC₅₀ (MGMT+/MMR+) divided by IC₅₀ (MGMT−/MMR+). The T1 indicates the efficacy of a compound in treating the targeted MGMT-cancer cells as compared to potential for the same compound to damage, or have adverse effects against, normal cells which are MGMT+cells, The higher the value of the TI, the safer the compound is. Further, the resistance index (RI) for each compound was calculated wherein RI is a measure of the efficacy of a compound in treating MGMT−/MMR− cancer cells, which give rise to TMZ resistance as compared to the same compound's efficacy against MGMT−/MMR+cells that are not believed to give rise to TMZ resistance. MMR RI (resistance index)=IC₅₀ (MGMT−/MMR−) divided by IC₅₀ (MGMT−/MMR+). The higher the value of the RI, the less likely that resistance to the compound will develop.

The IC₅₀ values of these compounds are shown in Table II, below.

TABLE II
IC₅₀ (μM) against LN229 cell lines

| | MGMT+ | MGMT | MGMT− | MGMT− | MGMT− | |
|---|---|---|---|---|---|---|
| Compound | MMR+ | MMR− | MMR+ | MMR− | TI | MGMT− RI |
| KL-50 | 587 ± 166 | 504 ± 55 | 24.7 ± 4.1 | 22.0 ± 6.9 | 24 | 0.9 |
| TMZ | 742 ± 468 | 1005 ± 107 | 8.3 ± 3.5 | 838 ± 100 | 89 | 101 |
| MTZ | 61.9 ± 4.7 | 50.8 ± 4.2 | 9.0 ± 0.4 | 6.9 ± 0.7 | 6.9 | 0.8 |
| CCNU | 21.6 ± 3.2 | 26.8 ± 7.3 | 6.3 ± 0.3 | 4.2 ± 0.2 | 3.4 | 0.7 |

TMZ, which is of known efficacious use for the treatment of cancers deficient in MGMT, was found to be essentially inactive in MGMT−/MMR− cells and this MMR deficiency is known to lead to tolerance of lesions and resistance to TMZ. MTZ and CCNU, although effective against MGMT−/MMR− cells, have a poor TI due to their efficacy against MGMT+cells and are more likely to have adverse effects in normal cells. KL-50 demonstrated exquisite sensitivity in MGMT−/MMR− cells, with limited activity in MGMT+cells, thereby exhibiting a superior TI and RI for MGMT-cells.

The activity of KL-50 in MGMT-LN229 cells engineered to lack expression of other key MMR proteins including MSH6, MLH1, PMS2, and MSH3 was also confirmed. Western blotting was performed in LN229 MGMT+/−cells with stable expression of shRNA targeting MSH6, MLH1, PMS2, or MSH3 to confirm depletion of the shRNA targets. In shMSH6 cells, there was reduced expression of MSH2, and in shMLH1 cells, there was loss of PMS2, due to destabilization in the setting of loss of their heterodimeric partners. GAPDH serves as loading control. Short-term viability assay curves for TMZ in LN229 MGMT+/−, MMR+/shMSH6 cells. Short-term viability assay curves for KL-50 in LN229 MGMT+/−, MMR+/shMSH6 cells. Short-term viability assay curves for TMZ in LN229 MGMT+/−, MMR+/shMLH1 cells. Short-term viability assay curves for KL-50 in LN229 MGMT+/−, MMR+/shMLH1 cells. Short-term viability assay curves for TMZ in LN229 MGMT+/−, MMR+/shPMS2 cells. Short-term viability assay curves for KL-50 in LN229 MGMT+/−, MMR+/shPMS2 cells. Short-term viability assay curves for TMZ (la) in LN229 MGMT+/−, MMR+/shMSH3 cells. Short-term viability assay curves for KL-50 in LN229 MGMT+/−, MMR+/shMSH3 cells.

TABLE III
IC₅₀ (μM) against LN229 cell lines

| | | MGMT+ | MGMT | MGMT− | MGMT− | MGMT− | MGMT− |
|---|---|---|---|---|---|---|---|
| shRNA | Compound | MMR+ | MMR− | MMR+ | MMR− | TI | RI |
| None | TMZ | 697 ± 60 | — | 5.1 ± 0.8 | — | 137 | — |
| None | KL-50 | 285 ± 46 | — | 13.2 ± 0.8 | — | 22 | — |
| shMSH6 | TMZ | — | 929 ± 201 | — | 950 ± 68 | — | 186 |
| shMSH6 | KL-50 | — | 362 ± 41 | — | 6.6 ± 0.4 | — | 0.5 |
| shMLH1 | TMZ | — | 769 ± 100 | — | 917 ± 85 | — | 180 |
| shMLH1 | KL-50 | — | 252 ± 28 | — | 7.8 ± 0.3 | — | 0.6 |
| shPMS2 | TMZ | — | 700 ± 58 | — | 921 ± 190 | — | 181 |
| shPMS2 | KL-50 | — | 215 ± 16 | — | 5.4 ± 0.3 | — | 0.4 |
| shMSH3 | TMZ | — | 649 ± 32 | — | 3.1 ± 0.6 | — | 0.6 |
| shMSH3 | KL-50 | — | 328 ± 26 | — | 7.9 ± 0.3 | — | 0.6 |

In Table III, above, IC₅₀ values derived from short-term viability assays in LN229 MGMT+/−cells lines, +/− shRNA, treated with TMZ or KL-50.

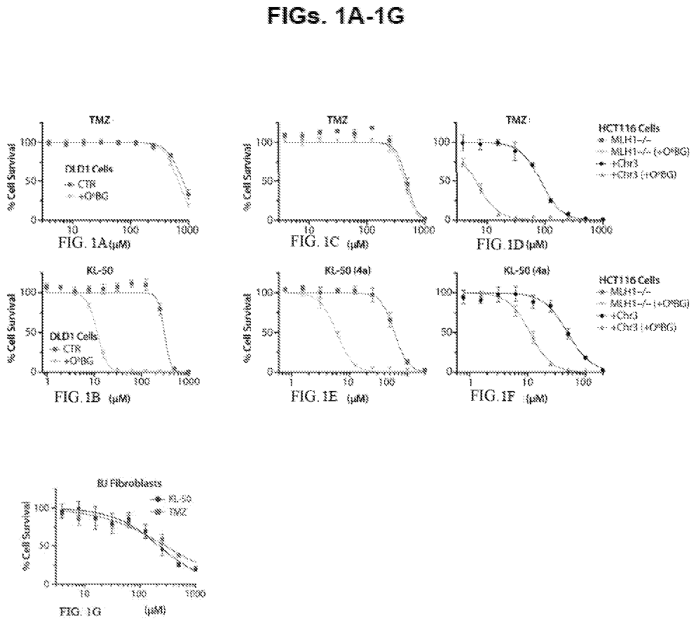

A similar pattern of activities was observed in several unique cell lines across different cancer types with intrinsic or induced loss of MGMT and/or MMR activity. For example, TMZ was inactive in DLD1 colorectal adenocarcinoma cells, which possess MGMT but lack functional MMR (MSH6−) with or without induced depletion of MGMT using O⁶-benzylguanine (O⁶BG; FIG. 1A). In contrast, KL-50 was toxic to these cells, but only after O⁶BG-induced MGMT depletion (FIG. 1B). TMZ was inactive in HCT116 colorectal cancer cells, which lack the MMR protein MLH1, regardless of MGMT levels (FIG. 1C). Restoration of MMR activity via complementation with chromosome 3 containing MLH1 resulted in the enhanced sensitivity to TMZ, which was further potentiated by MGMT depletion (FIG. 1D). In contrast, KL-50 induced selective tumor cell killing specifically in the setting of O⁶BG-induced MGMT suppression, in both MLH1-deficient cells (FIG. 1E) and MLH1-complemented cells (FIG. 1F). MMR status and O⁶BG-induced loss of MGMT expression was confirmed by western blot analysis.

The cytotoxicity of KL-50 and TMZ in normal human fibroblast cells were determined. No increase in toxicity was observed with KL-50 (FIG. 1G).

### Example 7: Clonogenic Survival Assays

In vitro clonogenic survival assays (CSAs) were performed using four isogenic LN229 glioblastoma cell lines.

The anti-cancer activity of KL-50, as compared to that of known anti-cancer agents TMZ and CCNU, was determined through performing these clonogenic survival assays.

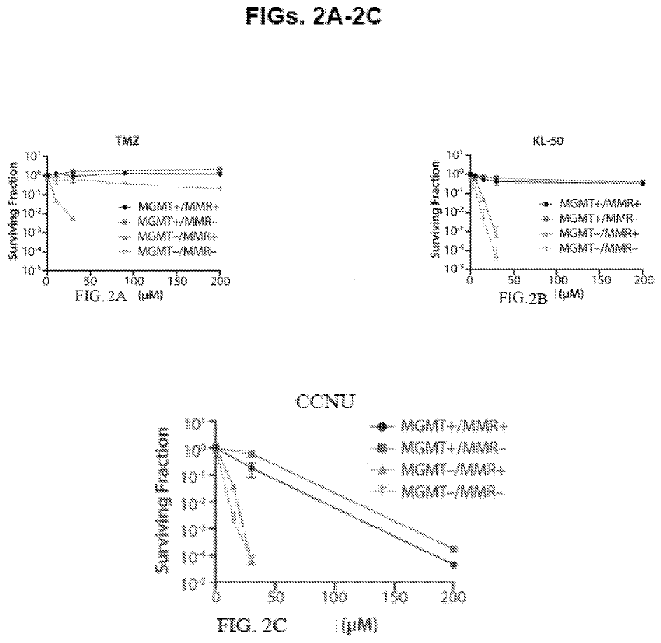

TMZ possessed negligible activity in MGMT+LN229 cells, irrespective of MMR status, and induced robust tumor cell killing in MGMT−, MMR+cells that was abolished in isogenic cells lacking MMR (FIG. 2A). CCNU was effective in MMR− cells but was cytotoxic to MGMT+cells (FIG. 2C). In contrast, KL-50 demonstrated robust antitumor activity in MGMT-cells, independent of MMR status, with minimal toxicity to MGMT+cells at doses up to at least 200 μM (FIG. 2B).

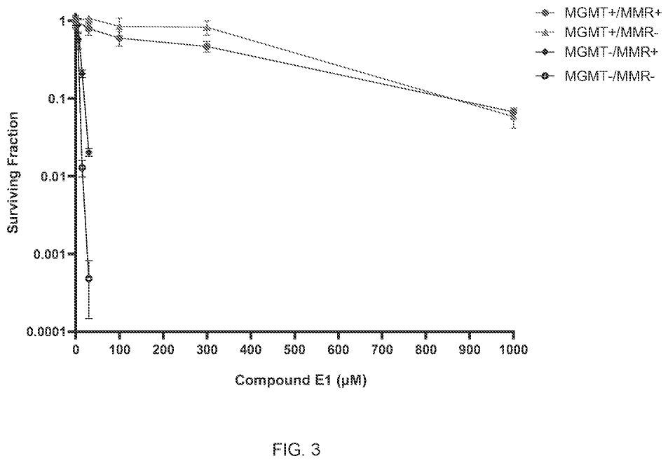

In addition, compound E1 was tested in the CSA assay, the results of which are shown in FIG. 3 which shows the surviving fraction of MGMT+ and MGMT-cells (both MMR proficient and MMR deficient) at increasing concentrations of E1. The surviving fraction of MGMT-cells dropped sharply at less than 100 μM of E1, showing that E1 severely inhibited the proliferation of the MGMT-cells, regardless of MMR status. In contrast, the surviving fraction of MGMT+cells remained at nearly 1.0 at the same concentration of E1 and remained above 0.1 at concentrations as high as 1000 μM of E1, showing that E1 had significantly less inhibition of the proliferation of MGMT+cells, regardless of MMR status.

### Example 8: IR Alkaline Comet Assay

The IR alkaline comet assay, adapted for ICL detection was performed to determine if ICLs were formed in MGMT-cells treated with KL-50. In this assay, cells were sequentially exposed to genotoxins and ionizing radiation, and then analyzed by single cell alkaline gel electrophoresis. Attenuation of the IR-induced comet tail is indicative of ICL formation. In the absence of IR, TMZ (200 μM) and KL-50 (200 μM) both induced tailing in MGMT−/MMR+cells, while mitomycin C (MMC, 0.1 or 50 μM) did not. Exposure to 50 μM MMC for 2 h completely abolished the IR-induced comet tail, whereas exposure to 0.1 μM MMC (chosen to be ˜10-fold greater than the IC₅₀ for this drug, comparable to 200 μM KL-50 or TMZ) for 24 h caused a partial reduction in the IR-induced comet tail. TMZ (200 μM) did not reduce DNA migration following IR, in agreement with its known function as a monoalkylation agent with no known crosslinking activity. In contrast, KL-50 (200 μM) reduced the % DNA in the tail to levels similar to those seen for 0.1 μM MMC. A similar pattern of comet tail migration was observed for MMC and KL-50 in MGMT−/MMR− cells, which supports an MMR-independent crosslinking mechanism.

This assay was performed at varying time points (2-24 h) to assess the rates of ICL formation in MGMT−/MMR− cells treated with KL-50, MTZ, or TMZ. The MTZ reduced DNA mobility within 2 h, consistent with the cell line selectivities above and literature reports that this agent rapidly forms ICLs by chloride displacement from other sites of alkylation.

TMZ did not induce a statistically significant decrease in DNA migration within 24 h. However, a time-dependent decrease in DNA mobility was observed in cells treated with KL-50, with the largest difference observed between 8 and 24 h, consistent with the reported half-life of O⁶FEtG (18.5 h). In the unirradiated samples, KL-50, MTZ and TMZ, all induced maximal damage at 2 h, which decreased over time, consistent with progressive DNA repair. Analysis of genomic DNA isolated from LN229 MGMT−/MMR+cells treated with KL-50 (200 μM) by denaturing gel electrophoresis demonstrated the presence of crosslinked DNA.

TMZ and MTIC showed no evidence of ICL induction. Similarly, linearized pUC19 plasmid DNA treated with KL-50 (100 μM) also possessed ICLs, with delayed rates of formation. Collectively, these data support a mechanism of action for KL-50 involving the slow generation of DNA ICLs in the absence of MGMT. Unrepaired primary KL-50 lesions convert to DNA ICLs in the absence of MGMT.

### Example 9: Alternate Mechanisms of Action for KL-50

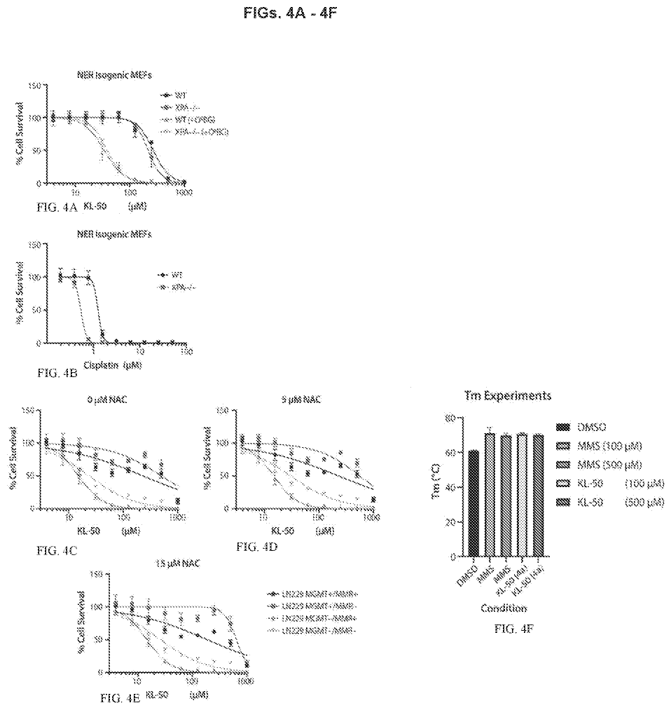

The potential for alternative mechanisms of action for KL-50 were probed implicating nucleotide excision repair (NER), base excision repair (BER), reactive oxygen species (ROS), and DNA duplex destabilization. Short term cell viability assays in isogenic mouse embryonic fibroblasts (MEFs) proficient or deficient in XPA, a common shared NER factor, revealed no differential sensitivity, either with or without O⁶BG-induced MGMT depletion (FIG. 4A). N7MeG lesions induced by TMZ are prone to spontaneous depurination, apurinic (AP) site formation, and single strand breaks (SSBs), which are all known BER substrates. To probe for potential differential induction of BER substrates by KL-50 compared to TMZ, in vitro supercoiled plasmid DNA assays were performed that measure the formation of AP sites. It was observed similar levels of spontaneous and enzyme-catalyzed SSBs from AP sites with KL-50 and TMZ, suggesting comparable levels of depurination. Co-treatment with increasing concentrations of the ROS scavenger N-acetylcysteine (NAC) did not rescue cell viability (FIG. 4B). Melting point analysis did not reveal any notable differences in DNA stability resulting from fluoroethylation compared to methylation (FIGS. 4C-4E). These data suggest that NER status, AP site induction, ROS, and altered DNA stability are peripheral or noncontributory to the effectiveness of KL-50.

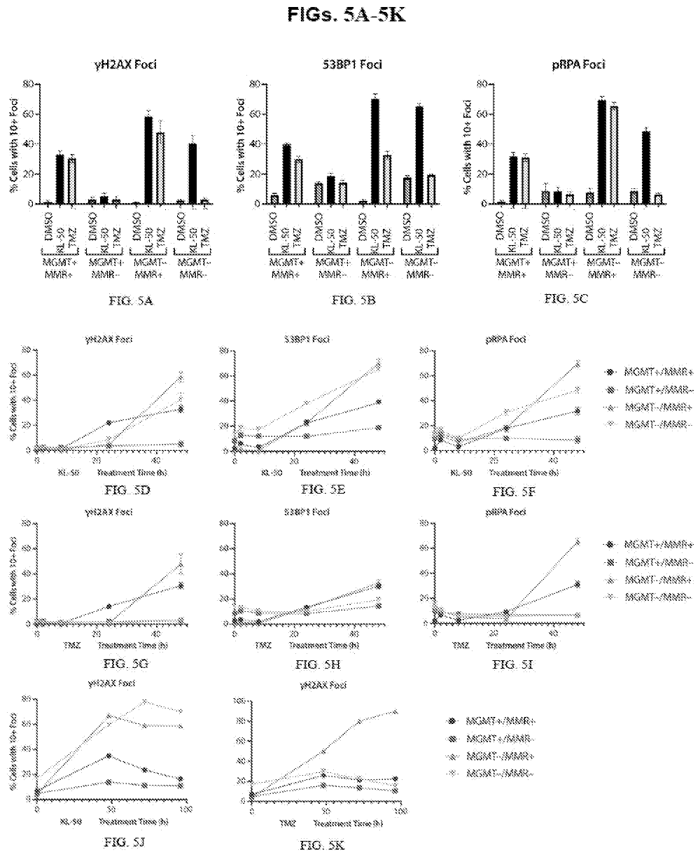

The profile of DDR activation across our four isogenic cell lines, after treatment with KL-50 or TMZ, was characterized. A prior finding that the ATR-CHK1 signaling axis is activated in response to TMZ-induced replication stress in MGMT-deficient cells prompted analysis of the phosphorylation status of CHK1 and CHK2 in LN229 MGMT+/− and MMR+/−cells. KL-50 induced CHK1 and CHK2 phosphorylation in MGMT-cells regardless of MMR status, whereas TMZ only induced phospho-CHK1 and —CHK2 in MGMT−/MMR+cells. Analysis of foci formation of the DDR factors phospho-SER139-H2AX (yH2AX), p53 binding protein 1 (53BP1), and phospho-SER33-RPA2 (pRPA) over the period of 2 to 48 h (FIGS. 5A-5C and FIGS. 5D-5I). KL-50 induced a maximal foci response at 48 h, specifically in MGMT-cells and irrespective of MMR status. TMZ induced a comparable response in MGMT-cells, but this was abolished in the absence of functional MMR, consistent with known MMR-silencing-based resistance. It was observed a reduced level of foci formation in MGMT+/MMR+cells that was absent in MGMT+/MMR− cells, suggesting an MMR-dependent DNA damage response in these cells. However, these foci dissipate at later timepoints (72-96 h; FIGS. 5J-5K), and they are not associated with appreciable cellular toxicity (as shown earlier in FIGS. 2A and 2B).

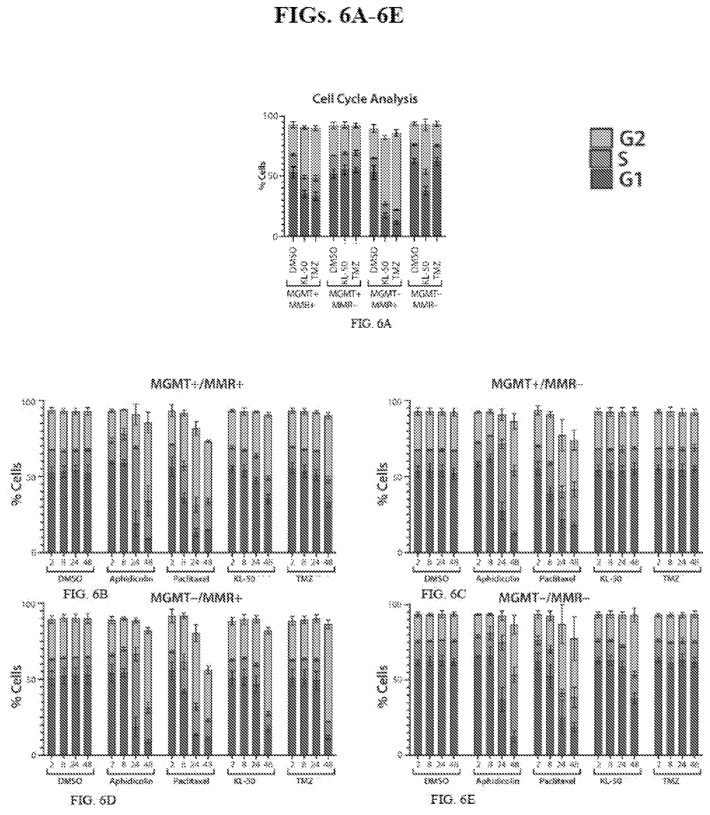

KL-50 induced increasing G2 arrest on progression from 24 to 48 h in MGMT−/MMR+cells, as determined by simultaneous analysis of DNA content based on nuclear (Hoechst) staining in the foci studies above (FIGS. 6A-6E). KL-50 induced an attenuated G2 arrest in MGMT−/MMR− cells, consistent with a role of MMR in the G2-checkpoint. This effect in MGMT−/MMR− cells was absent following TMZ treatment. Both TMZ and KL-50 induced a moderate G2 arrest in MGMT+/MMR+cells.

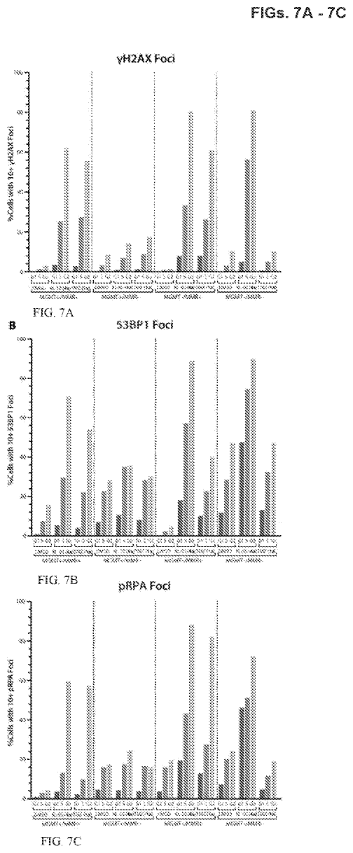

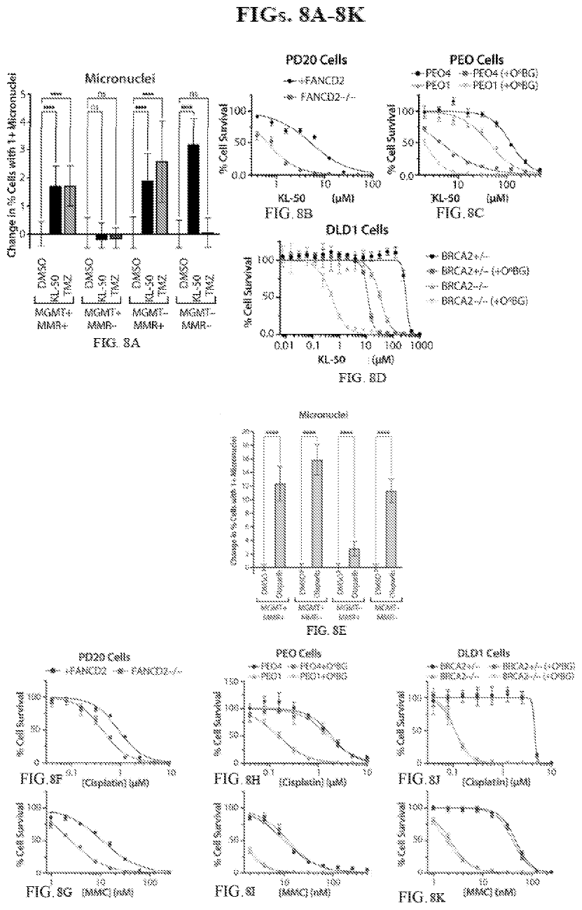

The levels of DDR foci across the individual cell cycle phases were quantified (FIGS. 7A-7C). KL-50 induced foci formation primarily in the S— and G2-phases of the cell cycle, which is consistent with replication blocking by ICLs. Foci increased in MGMT-G1 cells at 48 h, suggesting that a fraction of cells may progress through S-phase with unrepaired DNA damage. Consistent with this, a significant increase in micronuclei was observed at 48 h following KL-50 treatment, which was greatest in the MGMT−/MMR− cells (FIGS. 8A and 8E). TMZ displayed a similar pattern of foci induction in the S— and G2-phases, with smaller increases in G1-phase foci and micronuclei formation at 48 h in MGMT−/MMR+cells. In contrast, foci induction or micronuclei formation in MGMT−/MMR− cells exposed to TMZ was not observed. These findings are in agreement with the differential toxicity profiles of KL-50 and TMZ: KL-50 induces multiple successive markers of DNA damage and engagement of the DDR in MGMT-cells, independent of MMR status, whereas the effects of TMZ are similar in MGMT−/MMR+cells but absent in MMR− cells. Coupled with the ICL kinetics data presented above, these time-course data support a slow rate of ICL induction in situ by KL-50.

These foci data suggest that KL-50 induces replication stress (e.g., pRPA foci formation) and DSB formation (e.g., yH2AX and 53BP1 foci, which are known to follow the formation of ICLs). Consistent with this, BRCA2—and FANCD2-deficient cells are hypersensitive to KL-50 (FIGS. 8B-8D and 8F-8K). In two MGMT-proficient cell models, BRCA2 loss enhanced the toxicity of KL-50 following MGMT depletion via O⁶BG (FIGS. 8C-8D). FANCD2 ubiquitination by KL-50 was observed specifically in MGMT-cells, suggesting activation of the Fanconi anemia (FA) ICL repair pathway. As previously reported, TMZ also induced FANCD2 ubiquitination but only in MGMT−/MMR+cells.

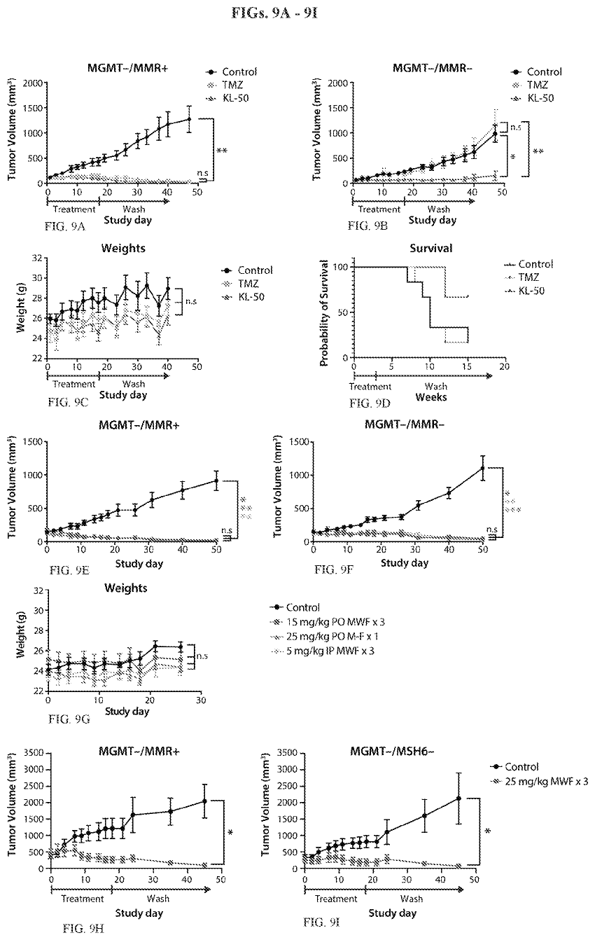

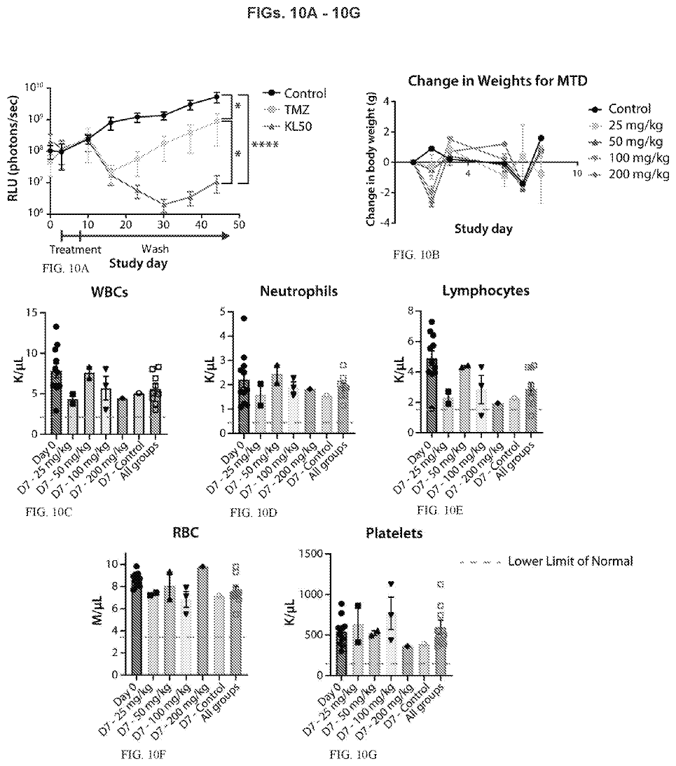

The activity of KL-50 and TMZ in vivo was evaluated using murine flank tumor models derived from the isogenic LN229 MGMT-cell lines The MGMT−/MMR+ and MGMT−/MMR− flank tumors were treated with KL-50 or TMZ (5 mg/kg MWF×3 weeks) as previously described for TMZ. TMZ suppressed tumor growth in the MGMT−/MMR+ tumors (FIG. 9A). KL-50 was statistically non-inferior to TMZ, despite a 17% lower molar dosage owing to its higher molecular weight. In the MGMT−/MMR− tumors, TMZ demonstrated no efficacy, while KL-50 potently suppressed tumor growth (FIG. 9B). KL-50 treatment resulted in no significant changes in body weight compared to TMZ or control (FIG. 9C). Representative Kaplan-Meier survival curves are shown in FIG. 9D with a greater than 5-week increase in median OS for KL-50 vs TMZ. KL-50 was effective and non-toxic using different dosing regimens (5 mg/kg, 15 mg/kg, 25 mg/kg), treatment schedules (MWF×3 weeks, M-F x 1 week), and routes of drug administration (PO, IP) in mice bearing MGMT−/MMR+ and MGMT−/MMR− flank tumors (FIGS. 9E-9G). KL-50 (25 mg/kg PO MWF×3 weeks) potently suppressed the growth of large (−350-400 mm³) MGMT−/MMR+ and MGMT−/MSH6—tumors (FIGS. 9H-9I). KL-50 (25 mg/kg IP M-F x 1 week) was also effective in an orthotropic, intracranial LN229 MGMT−/MMR− model, whereas TMZ only transiently suppressed tumor growth (FIG. 10A).

A focused maximum tolerated dose study revealed KL-50 is well-tolerated. Healthy mice were treated with escalating doses of KL-50 (0, 25, 50, 100, and 200 mg/kg x 1 dose), and monitored over time for changes in both weights and hematologic profiles. Mice in the higher dosage groups (100 or 200 mg/kg) experienced a greater than 10% weight loss after treatment administration, which regressed to baseline at the end of one week (FIG. 10B). Two of three mice in the 200 mg/kg treatment group became observably ill warranting euthanasia, but no evidence of toxicity was observed in the remaining cohorts. As the main dose limiting systemic toxicity of TMZ is myelosuppression, complete blood counts were measured for all mice on day 0 before treatment and subsequently on day 7 after drug administration. Overall, neutrophils and lymphocytes experienced the most significant drops in cell count, although all blood counts were within normal physiological ranges (defined as values falling within 2 SDs of the average for healthy mice) for all cohorts (FIGS. 10C-OG). Taken together, these data demonstrate the robust in vivo efficacy, systemic tolerability, and CNS penetrance of KL-50.

In the above examples, extensive characterization of KL-50 versus TMZ activity in vitro was performed which demonstrates that MGMT-cells, independent of MMR status, can be selectively targeted. While MGMT−/MMR− cells displayed no signs of DNA damage or DNA repair signaling in response to TMZ, robust, MMR-independent, activation of DNA damage checkpoint signaling, DNA repair foci formation, cell cycle arrest, and micronuclei formation following KL-50 treatment was found. Moreover, KL-50 retained its effectiveness in vivo in MMR-deficient flank and intracranial tumor models resistant to TMZ as well as in large MSH6-deficient tumors, a commonly lost MMR component reported in glioma patients.

Beyond MGMT-silenced recurrent glioma, other potential beneficial indications for selective targeting of cancer cells with KL-50 were demonstrated. MGMT silencing has been reported in 40% of colorectal cancers and 25% of non-small cell lung cancer, lymphoma, and head & neck cancers. MGMT mRNA expression is also reduced in subsets of additional cancer types, including breast carcinoma, bladder cancer, and leukemia. MMR loss, as reported by microsatellite instability, is a well-established phenomenon in multiple cancer types and leads to resistance to various standard of care agents. It therefore stands to reason that there are likely other subsets of MGMT−/MMR− tumors in both initial and recurrent settings that would be ideal targets for KL-50.

The data also suggest that KL-50 will display a higher therapeutic index in tumors with MGMT deficiency and impaired ICL repair, including HR deficiency. Specifically, it was demonstrated that FANCD2—and BRCA2-deficient cells are hypersensitive to KL-50, particularly in the setting of MGMT depletion. Remarkably, the therapeutic index (TI) of KL-50 in the DLD1 isogenic model, as measured by the ratio of IC₅₀ values in MGMT+/BRCA2+cells compared to MGMT−/BRCA2—cells, was ˜600-fold, vastly larger than canonical crosslinking agents such as cisplatin (42-fold) or MMC (26-fold). A similar amplification of the TI was seen in the PE01/4 model with KL-50 (62-fold) vs. cisplatin (13-fold) or MMC (7-fold). HR-related gene mutations have been detected in a substantial number of tumors across multiple cancer types (17.4% in 21 cancer lineages) and novel methods have been developed to assess for tumor-associated HR deficiency. Thus, in the modem era of molecular precision medicine, the biomarker-guided use of KL-50 in individual cancers could result in therapeutic indices and exquisite tumor sensitivities previously only observed with synthetic lethal interactions targeting DNA repair proteins. Finally, many possibilities can be envisaged for combination studies of KL-50 with DNA repair inhibitors such as checkpoint kinase inhibitors or potentially immunotherapy in the setting of MMR mutations. Further, the above findings indicate profound clinical implications for patients with recurrent MGMT-methylated glioma, of which up to half acquire TMZ resistance via loss of MMR. As demonstrated above, KL-50 is uniquely designed to fill this therapeutic void. In addition, because KL-50 may be rapidly phased into clinical trials and readily amenable to derivatization for improved drug pharmacokinetic properties, such enhanced as CNS penetration, based on prior work with the imidazotetrazine scaffold. More broadly, incorporating the rates of DNA modification and DNA repair pathways in therapeutic design strategies may lead to the development of additional selective chemotherapies.

### Biological Materials

Cell Culture. LN229 MGMT- and MGMT+ cell lines were a gift from B. Kaina (Johannes Gutenberg University Mainz, Mainz, Germany) and grown in DMEM with 10% FBS (Gibco). DLD1 BRCA2+/− and BRCA2−/− cell lines (Horizon Discovery, Cambridge, UK) were grown in RPMI 1640 with 10% FBS. HCT116 MLH1−/− and HCT116+Chr3 cell lines were a gift from T. Kunkel (National Institute of Environmental Health Sciences, Durham, NC) and grown in DMEM with 10% FBS, with 0.5 μg/mL G418 (Sigma) for HCT116+Chr3 cells. PD20 cell lines complemented with empty vector (+EV), wildtype FANCD2 (+FD2), or K561R ubiquitination-mutant FANCD2 (+KR) were a gift from G. Kupfer and P. Glazer (Yale University, New Haven, CT) and growth in DMEM with 10% FBS. PEO1 and PEO4 cell lines were a gift from T. Taniguchi (Fred Hutchinson Cancer Research Center, Seattle, WA) and were grown in DMEM with 10% FBS. BJ fibroblasts (normal human fibroblast cells) were purchased from ATCC (CRL-2522) and grown in DMEM with 10% FBS. NER isogenic MEFs were a gift from F. Rogers (Yale University, New Haven, CT) and were grown in DMEM with 10% FBS. All human cell lines were validated by short tandem repeat profiling (excluding BJ fibroblasts which were used within 6 passages of receiving from ATCC) and confirmed negative for mycoplasma by quantitative RT-PCR.

MMR Protein shRNA Knockdown. pGIPZ lentiviral shRNA vectors targeting MSH2, MSH6, MLH1, PMS2, and MSH3 were purchased from Horizon Discovery (Table IV). Lentiviral particles were produced in HEK293T cells via co-transfection with lentiviral shRNA plasmid, pCMV-VSV-G envelope plasmid (Addgene, #8454) and psPAX2 packaging plasmid (Addgene, #12260), using Lipofectamine 3000 Reagent (Invitrogen, L3000001) per manufacturer's protocol. Viral particles were harvested 48 h post-transfection and used to transduce LN229 MGMT+/−cells in the presence of 8 μg/mL polybrene. Selection of pooled cells with lentiviral expression was established with 1 μg/mL puromycin 48 h post-transduction for 3 to 4 days. Single cell cloning was performed by limiting dilution and protein knockdown was confirmed by western blotting.

TABLE IV
pGIPZ Lentivral shRNA Vectors for MMR protein knockdown

| Protein | pGIPZ Human Lentiviral | |
|---|---|---|
| Target | shRNA Clone | Mature Antisense Sequence |
| MSH2 | RHS4430-200305416 | TTACTAAGCACAACACTCT |
| | | (SEQ ID NO: 1) |
| MSH6 | RHS4430-200281418 | TACACATTACTTTGAATCC |
| | | (SEQ ID NO: 2) |
| MLH1 | RHS4430-200268977 | AACTGAGAAACTAATGCCT |
| | | (SEQ ID NO: 3) |
| PMS2 | RHS4430-200253216 | TTCACAGCTACATCAACCT |
| | | (SEQ ID NO: 4) |
| MSH3 | RHS4430-200158125 | TTTCTTGCAAATGCATTCG |
| | | (SEQ ID NO: 5) |

Biological Methodology:

Short-term Cell Viability Assay. Cells were seeded in 96-well plates at 1000 or 2000 cells/well and allowed to adhere at 23° C. for 60 min and then incubated overnight at 37° C. Cells were treated with indicated concentrations of compounds in triplicate for 4-6 days prior to fixation with 3.7% paraformaldehyde and nuclear staining with 1 μg/mL Hoechst 33342 dye. Cells were imaged on a Cytation 3 imaging reader (BioTek) and quantified using CellProfiler software. For in vitro short-term growth delay experiments, IC₅₀ values were determined from the nonlinear regression equation, [inhibitor] vs normalized response with variable slope.

Clonogenic Cell Survival Assay. Cells of each line were pretreated with the test drug in culture for 48-72 hours at the specified dilutions. The cells were then immediately seeded in six-well plates in triplicate at three-fold dilutions, ranging from 9000 to 37 cells per well. Depending on colony size, these plates were kept in the incubator for 10 to 14 days. After incubation, colonies were washed in phosphate-buffered saline (PBS) and stained with crystal violet. Colonies were counted by hand. Counts were normalized to plating efficiency of the corresponding treatment condition.

IR Alkaline Comet Assay. This assay was performed utilizing the CometAssay™ Kit (Trevigen) according to the alkaline assay protocol, with the addition of slide irradiation post-lysis. Cells were trypsinized, washed with 1×PBS, added to melted Comet LMAgarose (Trevigen), and spread on Trevigen CometSlides at a density of 1000 cells per sample in 50 μL. Lysis solution (Trevigen) with 10% DMSO was added overnight at 4° C. Slides were removed from lysis buffer and irradiated to 0 or 10 Gy using an XRAD 320 X-Ray System (Precision X-Ray) at 320 kV, 12.5 mA, and 50.0 cm SSD, with a 2 mm Al filter and 20 cm×20 cm collimator. Slides were then placed in alkaline buffer (200 mM NaOH, 1 mM EDTA) for 45 min, followed by electrophoresis in 850 mL alkaline buffer for 45 min at 4° C. Slides were washed and stained with SYBR gold (Invitrogen) per Trevigen assay protocol. Slides were imaged on a Cytation 3 imaging reader (BioTek), and comets were analyzed using CometScore 2.0 software (TriTek).

Genomic DNA Denaturing Gel Electrophoresis. Cells were trypsinized, washed with 1×PBS, and stored at −80° C. prior to processing. Genomic DNA was extracted with the DNeasy Blood & Tissue Kit (Qiagen) per kit protocol. A 0.7% agarose gel was prepared in 100 mM NaCl-2 mM EDTA (pH 8) and soaked in 40 mM NaOH-1 mM EDTA running buffer for 2 h. Genomic DNA (400 ng/well) was then loaded in 1×BlueJuice loading buffer (Invitrogen) and subjected to electrophoresis at 2 V/cm for 30 min, followed by 3 V/cm for 2 h. The gel was neutralized in 150 mM NaCl-100 mM Tris (pH 7.4) for 30 min, twice, and then stained with 1×SYBR Gold in 150 mM NaCl-100 mM Tris (pH 7.4) for 90 min. Imaging was performed on a ChemiDoc XRS+ Molecular Imager (Bio-Rad).

Plasmid Linearization Assay. To set up the linearization reactions, 20 units of EcoRI-HF (New England Biolabs) was mixed with 20 μg 2686 bp pUC19 vector DNA in CutSmart buffer (New England Biolabs), pH 7.9, in a total volume of 1000 μL for 30 min at 37° C. The CutSmart buffer contains 50 mM potassium acetate, 20 mM Tris acetate, 10 mM magnesium acetate, and 100 μg/mL BSA. The reacted DNA was then purified using PCR cleanup kit and quantified using the NanoDrop One (Thermo Fisher). The DNA was then stored at −20° C. before use in in vitro DNA cross-linking assays or melting temperature analysis.

In Vitro DNA Cross-linking Assays. Linearized pUC19 DNA, prepared as described above, was used for in vitro DNA cross-linking assays. For each condition, 200 ng of linearized pUC19 DNA (15.4 μM base pairs) was incubated with the indicated concentration of drug in 20 μL. Drug stock concentrations were made in DMSO such that each reaction contained a fixed 5% DMSO concentration. Reactions were conducted in 100 mM Tris buffer (pH 7.4). Cisplatin (Sigma) and DMSO vehicle were used as positive and negative controls, respectively. Reactions were conducted between 3-96 h at 37° C. The DNA was stored at −80° C. until electrophoretic analysis. For gel electrophoresis, DNA concentration was preadjusted to 10 ng/μL. Five microliters (50 ng) of the DNA solution was removed and mixed with 1.5 μL of 6×purple gel loading dye, no SDS, and loaded onto 1% agarose Tris Borate EDTA TBE gels. For denaturing gels, 5 μL (50 ng) of the DNA solution was removed and mixed with 15 μL of 0.2% denaturing buffer (0.27% sodium hydroxide, 10% glycerol, and 0.013% bromophenol blue) or 0.4% denaturing buffer (0.53% sodium hydroxide, 10% glycerol, and 0.013% bromophenol blue) in an ice bath. The mixed DNA samples were denatured at 4° C. for 5 min and then immediately loaded onto a 1% agarose Tris Borate EDTA (TBE) gel. All gel electrophoresis was conducted at 90 V for 2 h (unless otherwise noted). The gel was stained with SYBR Gold (Invitrogen) for 2 h.

EndoIV Depurination Assay. For each condition, 200 ng of supercoiled pUC19 DNA (15.4 μM base pairs) was incubated with the indicated concentration of drug in 20 μL for 3 hours. Drug stock concentrations were made in DMSO such that each reaction contained a fixed 5% DMSO concentration. Reactions were conducted in 100 mM Tris buffer (pH 7.4). For each EndoIV reaction, 50 ng of processed DNA was mixed with 20 units of EndoIV in NEBuffer 3.1 (New England Biolabs), pH 7.9, in a total volume of 20 μL for 16-20 h (unless otherwise noted) at 37° C. The NEBuffer 3.1 contained 100 mM sodium chloride, 50 mM Tris-HCl, 10 mM magnesium chloride, and 100 μg/mL BSA. For each negative control, 50 ng of processed DNA was mixed with NEBuffer 3.1, pH 7.9, in a total volume of 20 μL for 16-20 h (unless otherwise noted) at 37° C. Following completion of the experiment, the DNA was stored at −20° C. before electrophoretic analysis.

Melting Temperature Assay. Linearized pUC19 DNA (750 ng), prepared as described above, was incubated with the indicated concentration of either MMS or KL-50 adjusted in a final volume of 18 μL in 100 mM Tris buffer (pH 7.4) for 3 h. Drug stock concentrations were made in DMSO such that each reaction contained a fixed 5% DMSO concentration. Afterwards, 1 μL each of 20×SYBR Green dye (Invitrogen) and 20×ROX reference dye (Invitrogen) was added and melting temperature analysis was run on a StepOnePlus RT PCR System (Applied Biosciences) to generate melting temperature curves.

Immunofluorescence Foci Assays. High-throughput immunofluorescence foci assays were performed at the Yale Center for Molecular Discovery (YCMD). Cells were seeded at 2000 cells/well in black polystyrene flat bottom 384-well plates (Greiner Bio-One) and allowed to adhere overnight. Compound addition was performed utilizing a Labcyte Echo 550 liquid handler (Beckman Coulter), with 6 replicates per test condition and 12 replicates per control condition. Following drug incubation, cells were fixed and stained for phospho-SER139-H2AX (yH2AX), 53BP1, or phospho-SER33-RPA2 (pRPA) as follows.

γH2AXprotocol: Cells were fixed with 4% paraformaldehyde in 1×PBS for 15 min, washed twice with 1×PBS, incubated in extraction buffer (0.5% Triton X-100 in 1×PBS) for 10 min, washed twice with 1×PBS, and incubated in blocking buffer (Blocker Casein in PBS, Thermo Scientific+5% goat serum, Life Technologies) for 1 h. Mouse anti-phospho-histone H2A.X (Ser139) antibody (clone JBW301, Millipore, 05-636) was added 1/1000 in blocking buffer at 4° C. overnight. After washing with 1×PBS, cells were incubated with goat anti-mouse IgG (H+L) highly cross-adsorbed secondary antibody, Alexa Fluor 647 (Invitrogen, A-21236) 1/500 and with 1 μg/mL Hoechst nucleic acid dye in blocking buffer for 2 h, and then washed with 1×PBS.

53BP1protocol: Cells were fixed with 4% paraformaldehyde+0.02% Triton X-100 in 1×PBS for 20 minutes, washed twice with 1×PBS, and incubated in blocking buffer (10% FBS, 0.5% Triton X-100 in 1×PBS) for 1 h. Rabbit anti-53BP1 antibody (Novus Biologicals, NB100-904) was added 1/1000 in blocking buffer at 4° C. overnight. After washing with 1×PBS, cells were incubated with goat anti-rabbit IgG (H+L) highly cross-adsorbed secondary antibody, Alexa Fluor 647 (Invitrogen, A-21245) 1/500 and with 1 μg/mL Hoechst nucleic acid dye in blocking buffer for 2 h, and then washed with 1×PBS.

pRPA protocol: Cells were washed twice with 1×PBS on ice, incubated in extraction buffer (0.5% Triton X-100 in 1×PBS) for 5 min on ice, fixed with 3% paraformaldehyde+2% sucrose in 1×PBS for 15 min at 23° C., incubated again in extraction buffer for 5 min on ice, and incubated in blocking buffer (2% BSA, 10% milk, 0.1% Triton X-100 in 1×PBS) for 1 h at 23° C. Rabbit anti-phospho-RPA2 (S33) antibody (Bethyl Laboratories, A300-246A) was added 1/1000 in blocking buffer at 4° C. overnight. After washing 4 times with IF wash buffer (0.1% Triton X-100 in 1×PBS), cells were incubated with goat anti-rabbit IgG (H+L) highly cross-adsorbed secondary antibody, Alexa Fluor 647 (Invitrogen, A-21245) 1/500 and with 1 μg/mL Hoechst nucleic acid dye in blocking buffer for 1 h at 37° C. Cells were washed twice with IF wash buffer and twice with 1×PBS.

Imaging was performed on an InCell Analyzer 2200 Imaging System (GE Corporation) at 40× magnification. Twenty fields-of-view were captured per well. Foci analysis was performed using InCell Analyzer software (GE Corporation). Outer wells were excluded from analysis to limit variation due to edge effects.

Additional small scale immunofluorescence assays used for extended time course analysis of γH2AX foci were performed in Millicell EZSLIDE 8-well chamber slides (Millipore). Cells were seeded at 10,000 cells/well and allowed to adhere overnight. Following drug treatment, cells were fixed and stained for γH2AX as described above, without the addition of Hoechst dye. Slides were mounted with Vectashield Antifade Mounting Medium with DAPI (Vector Laboratories). Imaging was performed on a Keyence BZ—×800 fluorescence microscope at 40× magnification. Nine adjacent fields-of-view were captured per well and stitched together using a Fiji/ImageJ software plugin. Foci analysis was performed using Focinator v2 software.

Cell Cycle Analysis. Cell cycle analysis was performed using integrated Hoechst nucleic acid dye fluorescence intensity. Briefly, integrated Hoechst fluorescence intensity was log 2 transformed and histograms from DMSO-treated cells were used to identify the centers of the 2N and 4N DNA peaks. These values were used to normalize the 2N DNA peak to 1 and the 4N DNA peak to 2. Cells were then classified by normalized log 2 DNA content as G1 (0.75-1.25), S (1.25-1.75), or G2 (1.75-2.5) phase cells. The percentage of cells within each phase of the cell cycle was determined for each treatment condition. The three sets of Hoechst-stained cells corresponding to the three separate DNA foci stains were treated as three independent analyses.

Micronuclei Analysis. An automated image analysis pipeline was developed by YCMD using InCell Analyzer software to quantify micronuclei formation. Nuclei and micronuclei were segmented based on Hoechst nucleic acid dye staining channel. A perinuclear margin was applied around the nuclei to approximate the extent of the cytoplasm and identify micronuclei associated with the parent nucleus. Cells with nuclei associated with at least 1 micronucleus were considered positive.

Statistical analysis. Statistical analysis was performed using GraphPad Prism software. Data are presented as mean or median ±SD or SEM as indicated. For micronuclei assays, comparisons were made with one-way ANOVA and Sidak correction for multiple comparisons. For xenograft growth delay experiments, comparisons were made with Mann-Whitney test (for comparison of 2 groups) or Kruskal-Wallis test with FDR-adjusted p-values with Q set to 5% (for comparison of>3 groups). For xenograft survival analysis, Kaplan-Meier analysis was used to evaluate survival rate based on death or removal from study when body weight loss exceeded 20% of initial body weight.

Mouse Protocols.

Animals. All animal use was in accordance with the guidelines of the Animal Care and Use Committee (IACUC) of Yale University and conformed to the recommendations in the Guide for the Care and Use of Laboratory Animals (Institute of Laboratory Animal Resources, National Research Council, National Academy of Sciences, 1996).

Mouse Protocols for Flank Studies. A mouse tumor model was established by subcutaneously implanting human LN229 (MGMT−/MMR+) or LN229 (MGMT−/MMR−) cells. Cells were cultured as a monolayer in DMEM+10% FBS (Thermo Fisher) at 37° C. in a humidified atmosphere with 5% CO2 and passaged between one and three days prior to implantation and media was replaced every 2-3 days as needed to maintain cell viability. Cells were not allowed to exceed 80% confluency. On the day of implantation, cells were trypsinized, washed with complete media and pelleted by centrifugation at 1200 rpm for 5 minutes. The supernatant was decanted, and cells were washed three times with sterile PBS and pelleted by centrifugation. During the final centrifugation, viability was determined using trypan blue exclusion. Cells were resuspended in sterile PBS and diluted 1:1 in Matrigel (Coming, Cat #47743-716) for a final concentration of 5×10⁶ cells/100 μL. 5 million cells were injected into the flank of female nude mice (Envigo, Hsd:Athymic Nude-Fox1nu, 3-4 weeks age, 15 g). Once tumors reached a minimum volume of 100 mm³, mice were randomized and administered either KL-50; 5 mg/kg MWF×3 weeks), TMZ (5 mg/kg MWF×3 weeks), or vehicle (10% cyclodextrin) by oral gavage. Caliper measurements were obtained during the dosing period and at least two weeks following treatment. Mice were euthanized if body weight loss exceeded 20% or if tumor volume increased to greater than 2000 mm³. Kaplan-Meier analysis was used to evaluate survival rate based on death or removal from study.

In a second study, mice were randomized and administered either KL-50 or vehicle (10% cyclodextrin) by oral gavage or intraperitoneal injection on either M-F x 1 or MWF×3 cycles at 5, 15, or 25 mgs/kg. Caliper measurements were obtained during the dosing period and at least two weeks following treatment. Mice were euthanized if body weight loss exceeded 20% or if tumor volume increased to greater than 2000 mm³.

The third study involved MGMT−/MMR+ and MGMT−/MSH6—(shMSH6) LN229 cells. Mice tumors were allowed to grow to a larger average starting volume of −350 mm³ before they were randomized and administered either KL-50 (25 mg/kg MWF×3 weeks) or vehicle (10% cyclodextrin) by oral gavage. Caliper measurements were obtained during the dosing period and at least two weeks following treatment. Mice were euthanized if body weight loss exceeded 20% or if tumor volume increased to greater than 3000 mm³.

Mouse Protocol for Intracranial Study. LN229 MGMT−/MMR− cells stably expressing firefly luciferase (lentivirus-plasmids from Cellomics Technology; PLV-10003), were injected intracranially using a stereotactic injector. Briefly, 1.5 million cells in 5 pl PBS were injected into the brain and the mice were imaged weekly using the IVIS Spectrum In Vivo Imaging System (PerkinElmer) according to the manufacturer's protocol. Images were taken on a weekly basis and acquired 10 min post intraperitoneal injection with d-luciferin (150 mg/kg of animal mass). Tumors were allowed to grow to an average of 1.0×10⁸RLU before randomization and treated with 5 continuous days of P.O treatment with 10% cyclodextrin vehicle control, TMZ (25 mg/kg M-F x 1 week) or KL-50 (25 mg/kg M-F x 1 week). Quantification of BLI flux (photons/see) was made through the identification of a region of interest (ROI) for each tumor.

### Materials and Methods

General Chemical Experimental Procedures. All reactions were performed in single-neck, flame dried round-bottom flasks fitted with rubber septa under a positive pressure of argon, unless otherwise specified. Air- and moisture-sensitive liquids were transferred via syringe or stainless-steel cannula. Organic solutions were concentrated by rotary evaporation at 31° C., unless otherwise noted. Flash-column chromatography was performed as described by Still et al., employing silica gel (SiliaFlash® P60, 60 A, 40-63 μm particle size) purchased from Silicycle (Quebec, Canada). Analytical thin-layered chromatography (TLC) was performed using glass plates pre-coated with silica gel (250 μm, 60 A pore size) embedded with a fluorescent indicator (254 nm). TLC plates were visualized by exposure to ultraviolet (UV) light.

Chemical Materials. Commercial solvents, chemicals, and reagents were used as received with the follow exceptions. Dichloromethane, tetrahydrofuran, and toluene were purified according to the method described in A. B. Pangborn, M. A. Giardello, R. H. Grubbs, R. K. Rosen, F. J. Timmers, Safe and Convenient Procedure for Solvent Purification. Organometallics, 1518-1520 (1996). Triethylamine was distilled from calcium hydride under an atmosphere of nitrogen immediately prior to use. N,N-Di-iso-propylethylamine was distilled from calcium hydride under argon immediately prior to use. The diazonium, the imidazolyl triazene, the imidazolyl triazene, the imidazolyl triazene, and the imidazolyl triazene were synthesized according to published procedures.

Temozolomide (TMZ), lomustine (CCNU), O⁶-benzylguanine (O⁶BG), doxorubicin, and olaparib were purchased from Selleck Chemicals. Mitozolomide (MTZ) was purchased from Enamine. Methylmethane sulfonate (MMS) was purchased from Alfa-Aesir.

Mitomycin C (MMC), N-ethylmaleimide (NEM), N-acetyl-L-cysteine (NAC), and cisplatin were purchased from Sigma. TMZ (100 mM stock), O⁶BG (100 mM stock), MTZ (100 mM stock), MMS (500 mM stock) and NAC (100 mM stock) were dissolved in DMSO and stored at −80° C. MMC (10 mM stock), CCNU (100 mM stock), doxorubicin (10 mM stock), and olaparib (18.3 mM stock) were dissolved in DMSO and stored at −20° C. NEM (400 mM stock) was dissolved in EtOH and stored at −20° C. Cisplatin (5 mM stock) was dissolved in H₂O and stored at 4° C. for up to 7 days.

KL-50 was synthesized as follows.

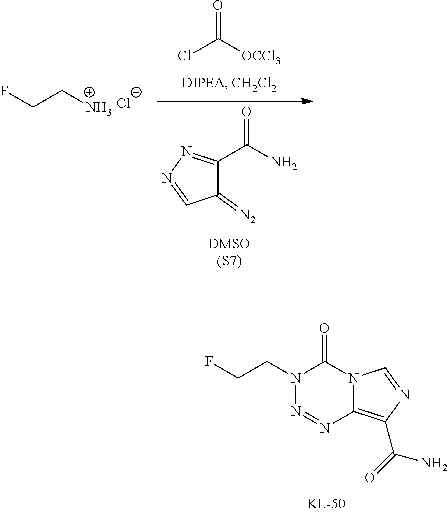

A mixture of fluoroethylamine hydrochloride (3.32 g, 33.3 mmol, 1 equiv), and N,N-di-iso-propyl ethylamine (12.2 mL, 70.0 mmol, 2.10 equiv) in dichloromethane (80 mL) was added dropwise via syringe pump over 45 min to a solution of diphosgene (2.40 mL, 20.0 mmol, 0.60 equiv) in dichloromethane (80 mL) at 0° C. (CAUTION: Gas evolution.). Upon completion of the addition, the cooling bath was removed, and the reaction mixture was allowed to warm to 23° C. over 15 min. The warmed product mixture was immediately transferred to a separatory funnel. The organic layer was washed sequentially with 1 N aqueous hydrochloric acid solution (100 mL, precooled to 0° C.) and saturated aqueous sodium chloride solution (100 mL, precooled to 0° C.). The washed organic layer was dried over magnesium sulfate. The dried solution was filtered, and the filtrate was concentrated (330 mTorr, 31° C.). The unpurified isocyanate so obtained was used directly in the following step.

The unpurified isocyanate obtained in the preceding step (nominally 16.7 mmol, 1.75 equiv) was added dropwise via syringe to a solution of the diazonium S7 (1.31 g, 9.54 mmol, 1 equiv) in dimethyl sulfoxide (10 mL) at 23° C. Upon completion of the addition, the reaction vessel was covered with aluminum foil. The reaction mixture was stirred for 16 h at 23° C. The product mixture was concentrated under a stream of nitrogen. The residue obtained was suspended in dichloromethane and purified by automated flash-column chromatography (eluting with 100% dichloromethane initially, grading to 5% methanol-dichloromethane, linear gradient) to provide KL-50 as a white crystalline powder (840 mg, 39% based on the diazonium S7).

¹H NMR (400 MHz, DMSO-d₆) δ 8.85 (s, 1H, H6), 7.83 (s, 1H, NH), 7.70 (s, 1H, NH), 4.82 (dt, J=47.0, 4.9 Hz, 2H, H3b), 4.62 (dt, J=26.0, 4.7 Hz, 2H, H3a). ¹³C NMR (151 MHz, DMSO-d₆) δ 161.5 (Csa), 139.2 (C4), 134.2 (C9), 131.0 (Cs), 128.9 (C6), 80.8 (d, J=168.7 Hz, C3b), 49.1 (d, J=20.8 Hz, C3a). ¹⁹F NMR (376 MHz, DMSO-d₆) 6-222.66 (tt, J =47.0, 26.1 Hz). IR (ATR-FTIR), cm¹: 3459 (w), 3119 (m), 1736 (s), 1675 (s). HRMS-ESI (m/z): [M+H]+ calcd for [C7HsFN6O2]+227.0688, found 227.0676.

Polymorphs of KL-50 were also found. Single crystals of KL-50 suitable for X-ray analysis were obtained by vapor diffusion of dry benzene (3 mL, precipitating solvent) into a syringe filtered (Millipore Sigma, 0.22 μm, hydrophilic polyvinylidene fluoride, 33 mm, gamma sterilized, catalogue number SLGV033RS) solution of KL-50 (3.6 mg) in dry dichloromethane (3 mL, solubilizing solvent) at 23° C. This yielded two polymorphs of KL-50 designated Polymorph I (P21/n space group, CCDC number 2122008) and Polymorph II (Cc space group, CCDC number 2122009).

Polymorph I of KL-50 was prepared as follows. Low-temperature diffraction data ((>-scans) were collected on a Rigaku MicroMax-007HF diffractometer coupled to a Dectris Pilatus3R detector with Mo Ku (Q=0.71073 A) for the structure of 007c-21083. The diffraction images were processed and scaled using Rigaku Oxford Diffraction software (CrysAlisPro; Rigaku OD: The Woodlands, T X, 2015). The structure was solved with SHELXT and was refined against F² on all data by full-matrix least squares with SHELXL (Sheldrick, G. M. Acta Cryst. 2008, A64, 112-122). All non-hydrogen atoms were refined anisotropically. Hydrogen atoms were included in the model at geometrically calculated positions and refined using a riding model. The isotropic displacement parameters of all hydrogen atoms were fixed to 1.2 times the U value of the atoms to which they are linked (1.5 times for methyl groups). The full numbering scheme of compound 007c-21083 can be found in the full details of the X-ray structure determination (CIF), which is included as Supporting Information. CCDC number 2122008 (007c-21083) contains the crystallographic data.

TABLE V
Crystal data and structure refinement for Polymorph I of KL-50:

| | |
|---|---|
| Identification code | 007c-21083 |
| Empirical formula | C7H7FN6O2 |
| Formula weight | 226.19 |
| Temperature | 93(2) K |
| Wavelength | 0.71073 Å |
| Crystal system | Monoclinic |
| Space group | P2₁/n |
| Unit cell dimensions | a = 7.1861(5) Å |
| | b = 7.6918(5) Å |
| | c = 16.4546(12) Å |
| Volume | 903.19(11) Å³ |
| Z | 4 |
| Density (calculated) | 1.663 Mg/m³ |
| Absorption coefficient | 0.141 mm⁻¹ |
| F(000) | 464 |
| Crystal size | 0.200 × 0.200 × 0.020 mm³ |
| Crystal color and habit | Colorless Plate |
| Diffractometer | Dectris Pilatus 3R |
| Theta range for data | 2.927 to 31.467° |
| collection | |
| Index ranges | −9 <= h <= 10, −10 <= k <= |
| | 9, −23 <= 1 <= 19 |
| Reflections collected | 9836 |
| Independent reflections | 2539 [R(int) = 0.0300] |
| Observed reflections | 2135 |
| (I > 2sigma(I)) | |
| Completeness to theta = | 99.9% |
| 25.242° | |
| Absorption correction | Semi-empirical from equivalents |
| Max. and min. transmission | 1.00000 and 0.31047 |
| Solution method | SHELXT-2014/5 (Sheldrick, 2014) |
| Refinement method | SHELXL-2014/7 (Sheldrick, 2014) |
| Data / restraints / parameters | 2539 / 0 / 145 |
| Goodness-of-fit on F² | 1.050 |
| Final R indices [I > | R1 = 0.0358, wR2 = 0.0885 |
| 2sigma(I)] | |
| R indices (all data) | R1 = 0.0450, wR2 = 0.0929 |
| Largest diff. peak and hole | 0.360 and −0.250 e · Å⁻³ |

Polymorph 11 of KL-50 was prepared as follows. Low-temperature diffraction data ((o-scans) were collected on a Rigaku MicroMax-007HF diffractometer coupled to a Satumn994+ CCD detector with Cu Ka (k=1.54178 A) for the structure of 007b-21124. The diffraction images were processed and scaled using Rigaku Oxford Diffraction software (CrysAlisPro; Rigaku OD: The Woodlands, T X, 2015). The structure was solved with SHELXT and was refined against F² on all data by full-matrix least squares with SHELXL (Sheldrick, G. M. Acta Cryst. 2008, A64, 112-122). All non-hydrogen atoms were refined anisotropically. Hydrogen atoms were included in the model at geometrically calculated positions and refined using a riding model. The isotropic displacement parameters of all hydrogen atoms were fixed to 1.2 times the U value of the atoms to which they are linked (1.5 times for methyl groups). The full numbering scheme of compound 007b-21124 can be found in the full details of the X-ray structure determination (CIF). CCDC number 2122009 (007b-21 124) contains the crystallographic data.

TABLE VI
Crystal data and structure refinement for Polymorph II of KL-50

| Identification code | 007b-21124 |
|---|---|
| Empirical formula | C7H7FN6O2 |
| Formula weight | 226.19 |
| Temperature | 93(2) K |
| Wavelength | 1.54184 Å |
| Crystal system | Monoclinic |
| Space group | Cc |
| Unit cell dimensions | a = 6.6061(2) Å |
| | b = 23.1652(6) Å |
| | c = 11.9879(3) Å |
| Volume | 1824.52(9) Å³ |
| Z | 8 |
| Density (calculated) | 1.647 Mg/m³ |
| Absorption coefficient | 1.218 mm⁻¹ |
| F(000) | 928 |
| Crystal size | 0.200 × 0.020 × 0.020 mm³ |
| Crystal color and habit | Colorless Needle |
| Diffractometer | Rigaku Saturn 944 + CCD |
| Theta range for data | 3.816 to 66.573°. |
| collection | |
| Index ranges | −7 <= h <= 7, −27 <= k <= |
| | 27, −14 <= 1 <= 14 |
| Reflections collected | 31349 |
| Independent reflections | 3204 [R(int) = 0.0818] |
| Observed reflections | 2950 |
| (I > 2sigma(I)) | |
| Completeness to theta = | 100.0% |
| 66.573° | |
| Absorption correction | Semi-empirical from equivalents |
| Max. and min. transmission | 1.00000 and 0.59718 |
| Solution method | SHELXT-2014/5 (Sheldrick, 2014) |
| Refinement method | SHELXL-2014/7 (Sheldrick, 2014) |
| Data / restraints / parameters | 3204 / 2 / 291 |
| Goodness-of-fit on F² | 1.041 |
| Final R indices [I > | R1 = 0.0330, wR2 = 0.0770 |
| 2sigma(I)] | |
| R indices (all data) | R1 = 0.0381, wR2 = 0.0795 |
| Absolute structure parameter | 0.1(2) |

Chemistry Instrumentation. Proton nuclear magnetic resonance (¹H NMR) were recorded at 400 or 600 megahertz (MHz) at 23° C., unless otherwise noted. Chemical shifts are expressed in parts per million (ppm, δ scale) downfield from tetramethylsilane and are referenced to residual proton in the NMR solvent ((CD₃)SO(CHD₂), δ2.50). Data are represented as follows: chemical shift, multiplicity (s=singlet, d=doublet, t=triplet, q=quartet, m=multiplet and/or multiple resonances, b=broad, app=apparent), coupling constant in Hertz (Hz), integration, and assignment. Proton-decoupled carbon nuclear magnetic resonance spectra (¹³C NMR) were recorded at 150 MHz at 23° C., unless otherwise noted. Chemical shifts are expressed in parts per million (ppm, (scale) downfield from tetramethylsilane and are referenced to the carbon resonances of the solvent (DMSO-d6, 6 39.52). ¹H-¹H gradient-selected correlation spectroscopy (COSY), ¹H-¹³C heteronuclear single quantum coherence (HSQC), and ¹H-¹³C gradient-selected heteronuclear multiple bond correlation (gHMBC) were recorded at 600 MHz at 23° C., unless otherwise noted. Carbon-decoupled fluorine nuclear magnetic resonance spectra (¹⁹F NMR) were recorded at 396 MHz at 23° C., unless otherwise noted. Chemical shifts are expressed in parts per million (ppm, 6 scale) downfield from tetramethylsilane. Attenuated total reflectance Fourier transform infrared (ATR-FTIR) spectra were obtained using a Thermo Electron Corporation Nicolet 6700 FTIR spectrometer referenced to a polystyrene standard. Data are represented as follows: frequency of absorption (cm¹), intensity of absorption (s=strong, m=medium, w=weak, br=broad). Analytical liquid chromatography-mass spectroscopy (LCMS) was performed on a Waters instrument equipped with a reverse-phase Cis column (1.7 μm particle size, 2.1×50 mm). Samples were eluted with a linear gradient of 5% acetonitrile-water containing 0.1% formic acid≥100% acetonitrile containing 0.1% formic acid over 0.75 min, followed by 100% acetonitrile containing 0.1% formic acid for 0.75 min, at a flow rate of 800 μL/min. HRMS were obtained on a Waters UPLC/HRMS instrument equipped with a dual API/ESI high resolution mass spectrometry detector and photodiode array detector.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

### ENUMERATED EMBODIMENTS

The following enumerated embodiments are provided, the numbering of which is not to be construed as designating levels of importance:

Embodiment 1 provides a compound of formula (I):

wherein:

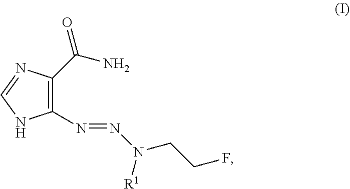

R¹ is selected from H, C₁₋₄ alkyl, hetero-substituted C₁₋₄ alkyl,

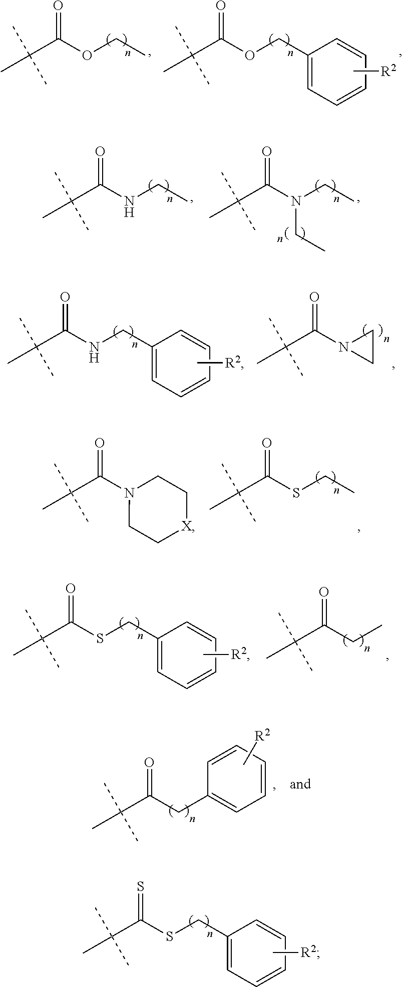

each occurrence of n is independently 0, 1, 2, 3, or 4;
X is independently selected from CH₂, NH, and 0;
R² is independently selected from H, C₁₋₄ alkyl, nitro, halogen, —OC₁₋₄ alkyl, —NHC₁₋₄ alkyl, —C(O)OC₁₋₄ alkyl, and C(O)NH— C₁₋₄ alkyl; and
“hetero-substituted C₁₋₄ alkyl” is independently selected from CH₂OR³, CH(OR³)R⁴, CH₂NR³R⁴, or CH₂NC(O)R³, and
R³ and R⁴ are each independently selected from C₁₋₄ alkyl; or a pharmaceutically acceptable salt thereof.

Embodiment 2 provides the compound of Embodiment 1, wherein R¹ is selected from the group consisting of hydrogen and C₁₋₄ alkyl.

Embodiment 3 provides the compound of any one of Embodiments 1-2, wherein R¹ is hydrogen.

Embodiment 4 provides the compound of any one of Embodiments 1-2, wherein R¹ is methyl.

Embodiment 5 provides a pharmaceutical composition comprising the compound of any one of Embodiments 1-4 and at least one pharmaceutically acceptable carrier.

Embodiment 6 provides a method of treating, ameliorating, and/or preventing cancer in a patient in need thereof, the method comprising administering to the patient a therapeutically-effective dose of:

(1) a compound, or a pharmaceutically acceptable salt thereof, of formula (I):
wherein:

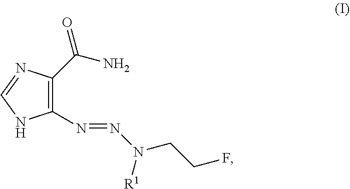

R¹ is selected from H, C₁₋₄ alkyl, hetero-substituted C₁₋₄ alkyl

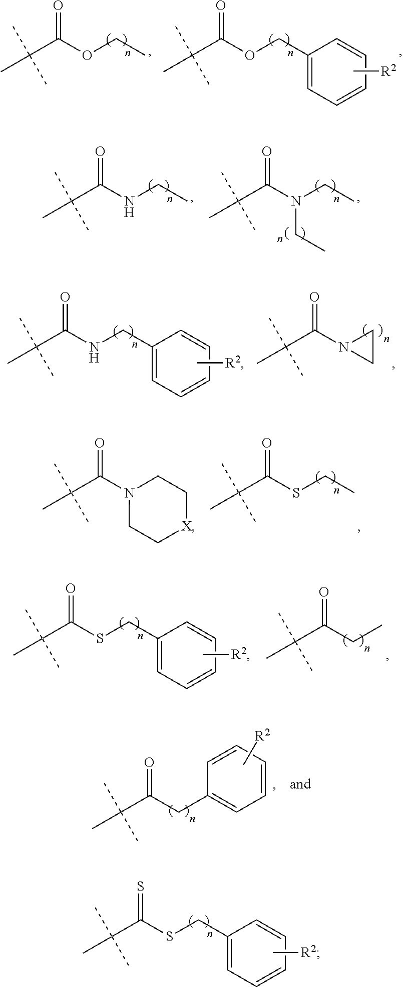

each occurrence of n is independently 0, 1, 2, 3, or 4;
X is independently selected from CH₂, NH, and 0;
R² is independently selected from H, C₁₋₄ alkyl, nitro, halogen, —OC₁₋₄ alkyl, —NHC₁₋₄ alkyl, —C(O)OC₁₋₄ alkyl, and C(O)NH— C₁₋₄ alkyl; and
“hetero-substituted C₁₋₄ alkyl” is independently selected from CH₂OR³, CH(0R³)R⁴, CH₂NR³R⁴, or CH₂NC(O)R³, and
R³ and R⁴ are each independently selected from C₁₋₄ alkyl;
   and/or
(2) a compound, or a pharmaceutically acceptable salt thereof, of formula (II):

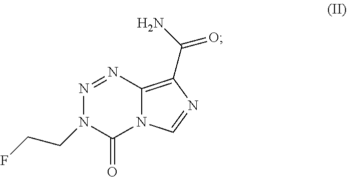 [SMILES: NC(=O)c1ncn2c(=O)n(CCF)nnc12]

wherein the cancer is MGMT-deficient.

Embodiment 7 provides the method of Embodiment 6, wherein R¹ is selected from the group consisting of hydrogen and C₁₋₄ alkyl.

Embodiment 8 provides the method of any one of Embodiments 6-7, wherein R¹ is hydrogen.

Embodiment 9 provides the method of any one of Embodiments 6-7, wherein R¹ is methyl.

Embodiment 10 provides the method of any one of Embodiments 6-9, further comprising the step of determining whether the cancer is MGMT-deficient.

Embodiment 11 provides the method of any one of Embodiments 6-10, wherein the cancer is a MGMT-deficient cancer.

Embodiment 12 provides the method of any one of Embodiments 6-11, wherein the cancer is MMR-deficient.

Embodiment 13 provides the method of any one of Embodiments 6-12, wherein the cancer is resistant to temozolomide.

Embodiment 14 provides the method of any one of Embodiments 6-13, wherein the MGMT-deficient cancer is selectively killed over normal tissue cells by the administering.

Embodiment 15 provides the method of any one of Embodiments 6-14, wherein normal tissue cells are not killed by the administering.

1. A compound of formula (I):
wherein: 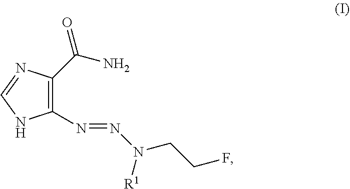
R¹ is selected from the group consisting of:
H, C₁₋₄ alkyl, CH₂OR³, CH(OR³)R⁴, CH₂NR³R⁴, CH₂NC(O)R³ 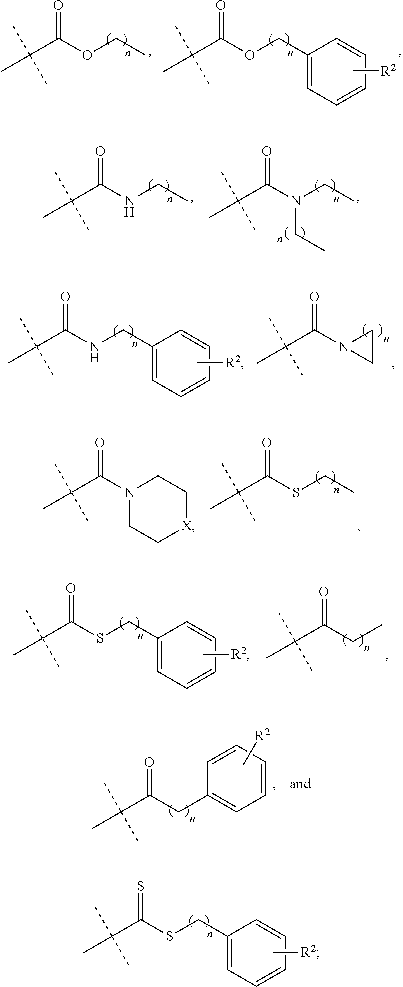
each occurrence of n is independently 0, 1, 2, 3, or 4;
X is independently selected from the group consisting of CH₂, NH, and 0;
R² is independently selected from the group consisting of H, C₁₋₄ alkyl, nitro, halogen, —OC₁₋₄ alkyl, —NHC₁₄ alkyl, —C(O)OC₁₋₄ alkyl, and —C(O)NH—C₁₋₄ alkyl; and
R³ is C₁₋₄ alkyl; and
R⁴ is C₁0.4 alkyl;
or a pharmaceutically acceptable salt thereof.

2. The compound of claim 1, wherein R¹ is selected from the group consisting of hydrogen and C₁₋₄ alkyl.

3. The compound of claim 2, wherein R¹ is hydrogen.

4. The compound of claim 2, wherein R¹ is methyl.

5. A pharmaceutical composition comprising the compound of claim 1 and at least one pharmaceutically acceptable carrier.

6. A method of treating or ameliorating an O⁶-methylguanine-DNA-methyltransferase (MGMT)-deficient cancer in a patient in need thereof, the method comprising administering to the patient a therapeutically-effective dose of:
(1) a compound, or a pharmaceutically acceptable salt thereof, of formula (I):
wherein: 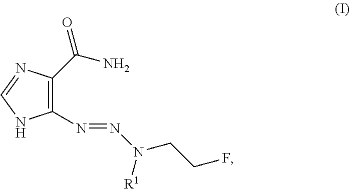
R¹ is selected from the group consisting of:
H, C₁₋₄ alkyl, CH₂OR³, CH(OR³R CI₂NR³R⁴, CH₂NC(O)R³ 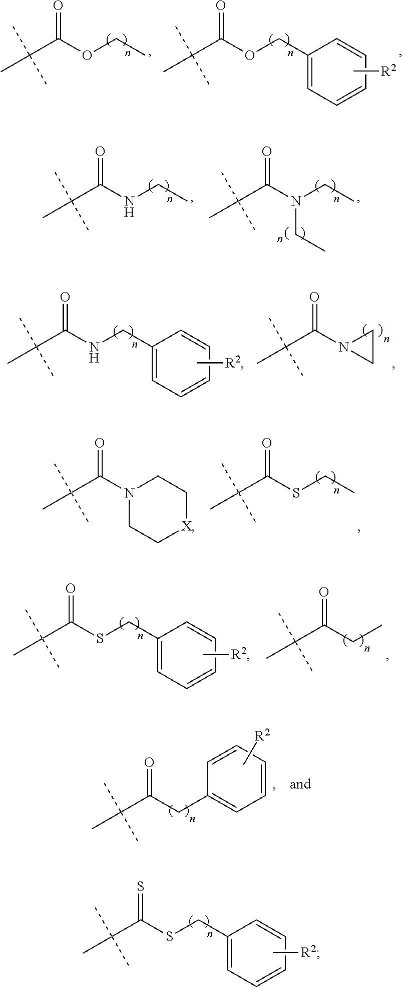
each occurrence of n is independently 0, 1, 2, 3, or 4;
X is independently selected from the group consisting of CH₂, NH, and 0;
R² is independently selected from the group consisting of H, C₁₋₄ alkyl, nitro, halogen, —OC1.4 alkyl, —NHC₁₋₄ alkyl, —C(O)OC₁₄ alkyl, and —C(O)NH— C₁₋₄ alkyl; and
R³ is C₁₋₄ alkyl; and
R⁴ is C₁₋₄alkyl; or
(2) a compound, or a pharmaceutically acceptable salt thereof, of formula (II):

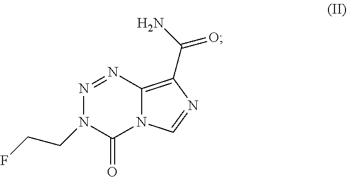 [SMILES: NC(=O)c1ncn2c(=O)n(CCF)nnc12]

7. The method of claim 6, wherein R¹ is selected from the group consisting of hydrogen and C₁₋₄ alkyl.

8. The method of claim 7, wherein R¹ is hydrogen.

9. The method of claim 7, wherein R¹ is methyl.

10. The method of claim 6, wherein the method further comprises, before the step of administering, a step of determining whether the cancer is MGMT-deficient.

11. (canceled)

12. The method of claim 6, wherein the cancer is mismatch repair (MMR)-deficient.

13. The method of claim 6, wherein the cancer is resistant to temozolomide.

14. The method of claim 6, wherein the MGMT-deficient cancer is selectively killed over normal tissue cells.

15. The method of claim 6, wherein normal tissue cells are not killed.
